(19) United States
(12) Patent Application Publication
(10) Pub. No.: US 2010/0222570 A1
(43) Pub. Date: Sep. 2, 2010

(54) Process for production of chlorinated sucrose based on hydrophobic affinity chromatography

(75) Inventors: Rakesh Ratnam, Karnataka (IN); Sundeep Aurora, Karnataka (IN); Arvind Mallinath Lali, Maharashtra (IN); Sandeep Bhaskar Kale, Maharashtra (IN)

Correspondence Address: THE NATH LAW GROUP, 112 South West Street, Alexandria, VA, 22314, US

(73) Assignee: PHARMED MEDICARE PVT. LTD., Mumbai, Maharashtra (IN)

(21) Appl. No.: 11/991,135

(22) PCT Filed: Aug. 29, 2006
(86) PCT No.: PCT/IN2006/000327

§ 371 Date: May 14, 2008

(30) Foreign Application Priority Data
Aug. 30, 2005 (IN) 1047/MUM/2005
Sep. 16, 2005 (IN) 1127/MUM/2005

(51) Int. Cl. C07H 1/06

(52) U.S. Cl. 536/127

(57) ABSTRACT

This invention relates to a process for selective capture, isolation and purification of chlorinated sucrose compounds, including chlorinated sucrose, their precursors and derivatives, including trichlorogalactosucrose (TGS), directly from chlorinated reaction mixture by column chromatography on adsorbents and under conditions which result in specific and selective affinity towards one or more of chlorinated sucrose compound. The process also integrates de-esterification of chlorinated sucrose esters adsorbed on the adsorbent while they are being treated with desorbent. The process also provides a novel approach to concentration and crystallization of TGS. The chlorinated sucrose derivatives, including TGS, thus isolated are substantially free from most impurities, salts and organic solvents. The process has high recovery of more than 95% in terms of desired chlorinated sucrose derivatives including TGS.

### TECHNICAL FIELD

The present invention relates to a novel process and a novel strategy for purification of the product 1-6-Dichloro-1-6-DIDEOXY-p-Fructofuranasyl-4-chloro-4-deoxy-galactopyranoside (TGS) or its intermediates or derivatives by affinity and/or hydrophobic adsorption chromatography from reaction mixture or solutions containing chlorinated sucrose compounds including TGS, their intermediates or derivatives.

### BACKGROUND OF THE INVENTION

Strategies of prior art methods of production of 4,1′,6′ trichlorogalactosucrose (TGS) predominantly involve chlorination of sucrose-6-ester by use of Vilsmeier-Haack reagent derived from various chlorinating agents such as phosphorus oxychloride, oxalyl chloride, phosphorus pentachloride etc, and a tertiary amide such as dimethyl formamide (DMF) or dimethyl acetamide to chlorinate Sucrose-6-ester, to form 6 acetyl 4,1′,6′ trichlorogalactosucrose. After the said chlorination reaction, the reaction mass is neutralized to pH 7.0-7.5 using appropriate alkali hydroxides of calcium, sodium, etc. to deesterify/deacetylate the 6 acetyl 4,1′,6′ trichlorogalactosucrose to form 4,1′,6′ trichlorogalactosucrose (TGS).

In addition to the above described process of production of TGS based on chlorination process, there are several alternative methods for TGS production, each of which produce process streams of varying composition depending on the process used containing one or more of TGS, its intermediates, derivatives, unreacted raw material, salts, catalysts and several other reactants involved in the reaction, and a problem common to all is the need of a more convenient and scalable industrial process for removal of difficult to remove constituents such as dimethylformamide (DMF) and isolation of one or more of the constituents of the reaction mixture individually or collectively in groups comprising TGS, TGS precursors, TGS derivatives and the like from closely related organic impurities and inorganic impurities.

Various prior art methods have been described for the removal of the tertiary amide including steam distillation, column chromatography, reverse osmosis, Agitated Thin Film Drying, etc. Further isolation of the chlorinated sucrose derivatives are accomplished by operations such as extractive purification, silica gel column chromatography, crystallization, etc.

This invention provides for a novel process based on column chromatography including hydrophobic affinity chromatography which is easy to operate, is scalable and efficient in achieving removal of impurities and isolation of desired chlorinated sucrose products.

Water is also needed to be removed from reaction mixtures at various stages in process of production of TGS, which is cumbersome for removal by hitherto known conventional processes of water removal. A need for developing an alternative process for water removal and concentration of chlorinated sucrose derivative/s was also felt and is dealt with in this invention.

### PRIOR ART

Various prior art processes have been described for the removal of the tertiary amide solvent, purification of TGS and protected or partially protected TGS precursors, and use methods such as steam distillation, column chromatography, reverse osmosis, agitated thin film drying, etc. Partially purified mixture thus obtained is subjected to further purification using operations such as extractive purification, silica gel column chromatography, crystallization, etc.

Prior art methods are available for purification of product TGS as well as other chlorinated sugars from deacylated reaction mixture by column chromatography using silica gel as adsorbent and eluants of increasing polarity as desorbents. Thin layer chromatography on silica gel is a well known public domain method for detecting TGS or its derivatives. Use of column chromatography for such separations from a reaction mixture and particularly use of silica gel as adsorbent and solvents of increasing polarity for purification of TGS from deacylated reaction mixture is also known in public domain for a long time. Several patents, expired as well as in-force have described them.

Thus, Mufti, et al. (1983), in U.S. Pat. No. 4,380,476 have mentioned in their description: “Alternatively, the success of the overall process according to the present invention will depend in part on the fact that TGS itself can be isolated without undue difficulty from the deacetylated mixture of chlorinated sucrose derivatives obtained. We have found that chromatography, e.g. on silica gel, will isolate TGS relatively simply. For example, elution of the deacylated mixture with a series of eluants of increasing polarity removes first the less polar by-products and then TGS, while more polar compounds remain bound. Mixtures of chloroform and acetone are particularly suitable: a 2:1 mixture followed by a 1:1 mixture is effective in isolating TGS in the 1:1 eluate. We prefer to chromatograph after deacylation, but chromatographic separation of TGS 6-acylate is also possible.” Examples of this have been described in example nos. 1 and 3 in this patent.

Khan, et al. (1992) in U.S. Pat. No. 5,136,031 in Example no. 3 have described that a solution of sucrose 6,4′-diacetate in pyridine, was treated with thionyl chloride in 1,1,2-trichloroethane, initially at 0.degree. C. for 0.5 h, and then at 95.degree. C. for 4 h. The reaction mixture was diluted with methylene chloride, washed with cold aqueous sodium carbonate and then with water. The organic layer was dried (Na.sub.2 SO.sub.4), concentrated by co-distillation with toluene, and then treated with 1M sodium methoxide in methanol (pH10.0) at room temperature for 4 h. T.I.C (ethyl acetate: acetone: water, 8:6:1) revealed sucralose as the major product, which was purified by silica gel chromatography and characterised by .sup.1H-NMRspectroscopy.

Dordick, et al. 1992 in U.S. Pat. No. 5,128,248 mentioned in the description that “resulting in a mixture of the 6-mono- and the 6,4′-diacylate. The two acylates can be separated e.g. by chromatography on a silica gel column, if required.” They have described in Example 6, a process of conversion of sucrose 6,4′-diacetate into Sucralose wherein from the deacylated reaction mixture, sucralose as the major product was purified by silica gel chromatography and characterized by .sup.1H-NMR spectroscopy.

Walkup et al., (1990), in U.S. Pat. No. 4,980,463 have mentioned that typically, the chlorinated products resulting from the chlorination of sucrose or its derivatives are purified and isolated by chromatographic techniques or by derivatization to form highly crystalline solids (e.g., peracetylation). The said patent however does not mention the type of chromatography and the adsorbent medium used for it.

U.S. Pat. No. 4,343,934 relates to the crystallization of TGS from an aqueous solution after silica gel chromatography for solid TGS, and then deionization of the reaction mixture using combination of ion exchange resins Amberlite IRA 35 and IRC 72. This is followed by two cycles of heating the remaining mother liquor, concentrating, adding seed crystals, and cooling. This followed by three cycles of crystallization provided an overall recovery of TGS from the syrup obtained after deacylating sucrose pentaacetate is 76.6%. It is important to note that the ion exchange adsorbent resins used in the said patent were intended to deionize the reaction mixture by specifically adsorbing the soluble ions not necessarily TGS.

Jenner et al. (1982) in U.S. Pat. No. 4,362,869 have described column chromatography for separation of trichlorinated ester. Here they have reported that the reaction mixture can conveniently be worked up by pouring it into water and extracting with an organic solvent such as dichloromethane. The extracts, when washed with acid and with base, dried and evaporated, yield a product which can be further purified by chromatography, for example on silica gel, to give a yield of the tri-chlorinated ester of approaching 80% with respect to starting content of 2,3,6,3′,4′-penta-O-acetylsucrose.

Chromatography has also been described for separation of TGS pentaacetate in Example no. 9 in this patent which describes that “This syrup was chromatographed on a column of silica gel, eluted with diethyl ether/40.degree.-60.degree. petroleum ether (4:1), to give TGS pentaacetate (1.2 g 78%) which was crystallized from ethanol and found to be identical with an authentic sample.”

U.S. Pat. No. 4,405,654 discloses the synthetic routes for synthesizing various halosucrose derivatives. The compounds are isolated by silica gel column chromatography. The patent also discloses the use of ionic resin for neutralization and deionization.

Rathbone et al., (1989) in U.S. Pat. No. 4,826,962 on “Tetrachlororaffinose and its use in the preparation of sucralose” has mentioned use of chromatographic methods wherein “The separation of the sucralose product may be achieved by any convenient steps, for example by evaporation and extraction into an organic solvent, by chromatographic techniques, or by selective crystallization from either the aqueous or the non-aqueous systems.” They have described in Example no. 4 that “The products were separated by chromatography and, in addition to sucralose, the presence of 6-chlorogalactose and TCR was detected.” The said patent however does not mention the type of chromatography and the adsorbent medium used for it.

U.S. Pat. No. 4,980,463 discloses processes for purifying TGS-6-benzoate including extraction, crystallization followed by recrystallization. This ester is then alkali hydrolyzed and neutralized using an ion exchange resin Amberlite IRC-50 in H+ form. Also shown is an extractive crystallization, which combines extraction and a first crystallization in a single step.

Prior art methods are also available for separation and purification of TGS-6-acetate from reaction mixtures. All of them have used either ion exchange resins or silica gel for chromatography. Thus, when claim nos. 1, 8, 9, 10 and 14 of Mufti, et al. (1983), in U.S. Pat. No. 4,380,476 are read with what has been mentioned in this specification in the detailed description that “We prefer to chromatograph after deacylation, but chromatographic separation of TGS 6-acylate is also possible.” makes it clear that isolation and purification of TGS-acetate directly from reaction mixture/process stream based on chlorination process, which produced several closely related chlorinated sucroses, was anticipated in the prior art by use of column chromatography, the adsorbents used for that purpose were either silica gel or ion-exchange resin chromatography or both, and the principle used was non-specific adsorption/desorption depending on differences in hydrophilic and hydrophobic properties of the molecules present in the solution chromatographed.

U.S. Pat. No. 5,128,248 described the use of silica gel for isolation of intermediates and for purification of TGS.

U.S. Pat. No. 5,298,611 discloses a steam stripping process for DMF removal from reaction mixture containing TGS.

U.S. Pat. No. 5,498,709 disclose a process in which TGS-Acetate is deacetylated prior to or after DMF removal and TGS is recovered by extraction and purified by crystallization.

U.S. Pat. No. 5,530,106 describes the removal of dimethyl formamide (DMF) by steam distillation or steam stripping from liquid mixture containing TGS-acetate followed by extraction of TGS-acetate and repetitive crystallization to get pure TGS-acetate. The product thus obtained was then recrystallized, hydrolyzed, passed through Amberlite IRC-50 ion exchange resin, followed by concentration, extraction, and finally crystallization to get pure TGS.

Catani, et. al., (1999) in U.S. Pat. No. 5,977,349 entitled “Chromatographic purification of chlorinated sucrose” described a polystyrene-based sodium sulfonic resin, crosslinked with 4% divinylbenzene, as adsorbent, and straight water as desorbent. An elution order: salt>Di's>6,6′>sucralose>6,1′, 6,>4,6,6′>Tet's is revealed. The separation process in the said patent thus describes the use of porous gel type sodium sulfonic acid based cation exchange resin and silica gel with water and organic solvent as desorbent, respectively. Further, the patent describes use of a fixed bed in radial flow or annular chromatography; continuous annular chromatography (CAC) simulated moving bed (SMB) chromatography. The patent also discloses the possibility of purifying esterified reaction mixture by radial process prior to hydrolysis and reverse phase chromatography for sucralose-6-acetate. In case of porous gel type cation exchange resin, the patent discloses use of 2% to 6% of divinyl benzene (DVB) as the adsorbent.

Catani et al (2006) in U.S. Pat. No. 7,049,435 have described processes for extractive purification of TGS from a process stream. Purification of chlorinated sucrose derivative/s by liquid extraction process requires repetitive extraction and back extraction steps using water and organic solvents. Multiple extraction and back extraction operations reduce the overall yield of the process due to finite water solubility of chlorinated sucrose derivative/s such as TGS. Also, the solvents used in the extraction processes carry considerable moisture which also lowers yields in crystallization steps.

Aqueous solutions of chlorinated sucrose derivative/s obtained from a chromatographic process, or any other purification method, requires removal of water for crystallization step. This is usually carried out using liquid-liquid extraction of the chlorinated sucrose derivatives, including TGS, into organic solvents or by distillation. Distillation to remove water from product is both time and energy intensive operation and also has adverse effect on the product quality because of longer exposure times to higher temperatures.

Alternatively, the hydroxyl group protected chlorinated sucrose such as TGS can be purified by extraction at good yields as the hydroxyl group protected TGS has low water solubility. This hydroxyl group protected TGS can be hydrolyzed chemically or enzymatically to give TGS. A typical chemical process generates salts and side products that further necessitate purification of chlorinated sucrose derivative/s including TGS by extraction or chromatography. The enzymatic process, on the other hand, requires presence of water for hydrolysis. Both these hydrolysis methods require water removal and hence the problem remains similar as stated above.

It may be noted that none of the patents using chromatography as method of separation has used non-ionic polymeric resins.

Thus, in all the prior art processes on column chromatography, conventional silica gel and ion exchange resins such as those based on polystyrene or polystyrene cross-linked with di-vinylbenzene and the like are used as adsorbent and all these methods are based on principle of relative differences of molecules with respect to hydrophilic-hydrophobic interactions with the ionic and/or polar adsorbents as well as the eluants. These differences are often very narrow for structurally closely similar molecular species, they result in overlapping areas in their elution curves. Net result is that it is often difficult to cleanly separate two closely related molecules in high enough yields and a large proportion is retrieved as mixtures in the eluants. Same problems of inadequate separation of closely related molecules arise in solvent extraction methods, in addition to problems of inadequate separation of molecular species due to partial miscibility of two solvents, need for repetitive extraction leading to large volume of solvents, which need to be recovered by high input of energy.

DMF removal by steam distillation or steam stripping is energy intensive for large volume applications as DMF is a relatively high boiling solvent. Further, steam distillation can degrade the product from which purification of TGS becomes more difficult and results in lower yield and purity.

Further, in all prior art processes, usually in a process involving chlorination process, wherever the reaction mass containing TGS-Acetate is hydrolyzed to form TGS in presence of DMF using alkali, the alkali rapidly degrades the expensive solvent DMF, which adds considerable cost per unit weight of TGS produced.

Prior art processes have left a large scope for further improvement in efficiency of the process and in the quality as well as yield of the recovered product.

### SUMMARY OF INVENTION

This invention embodies a surprisingly simple novel column chromatographic process, based on hydrophobic affinity column chromatography, to achieve from a process stream obtained in a process for production of TGS, in sequential steps on same or different columns and on same or different adsorbents, removal of DMF and inorganic impurities, isolation of TGS-esters including TGS-acetate, and TGS-arylate, and de-esterification of the said TGS-esters integrated on the column itself, isolation of TGS, concentration of TGS and dewatering of TGS. A further embodiment of the invented process includes a process for concentration of target product molecules from dilute solution to a composition having 5% or less of water in it.

A yet another embodiment of the process also includes regeneration of the adsorbents several times leading to better process efficiency as well as cost efficiency.

Affinity chromatography comprises use of an adsorbent surface that can display a degree of relative interacting ability such as affinity for the target molecule over some of the other components of the mixture. In the present invention the adsorbents used are typically rigid or gel type porous adsorbents made up of organic polymers of natural, synthetic and semi-synthetic origin. The adsorbent resins can also comprise of C2 to C18, straight chain or branched chain, containing molecules, or aromatic hydrophobic molecules deposited or grafted on the surface of adsorbents.

The process of this invention applies to all halogenated sucroses with appropriate modification and adaptations, although illustrated embodiments relate to a process as applied to process streams arising from a process of production of TGS.

In one embodiment, the process of the present invention relates to the capture, isolation and purification of chlorinated derivatives of sucrose, including TGS, by adsorption chromatography on a porous polymeric adsorbent matrix that displays some degree of selectivity for the desired chlorinated sucrose derivatives under operating conditions appropriate for desired interactions between the chlorinated derivatives of sucrose and the chosen adsorbent.

This embodiment provides the process for capture and purification of TGS and/or protected or partially deprotected TGS from the neutralized chlorinated reaction mass; and which comprises of,
a) bringing the pH adjusted chlorinated reaction mass into contact with an equilibrated rigid porous adsorbent matrix whereby the said chlorinated derivatives adsorb onto it, and
b) washing the adsorbed matrix to remove unadsorbed components of the reaction mass including DMF and salts, and
c) progressive and/or selective elution of the chlorinated derivatives from the matrix using an appropriate elution mobile phase, and
d) regenerating the adsorbent matrix for reuse.

Thus the process performs the capture and purification of chlorinated sucrose (including TGS) or its derivative (including TGS-acetate, TGS-benzoate and the like) with simultaneous removal of DMF and salts, and produces a product free from DMF and salts. The eluted chlorinated sucrose or its derivative is then polished using similar, or another adsorbent, in a second column to remove traces of most other impurities, and results in a product such as TGS, TGS-acetate, or TGS-benzoate, substantially free from all impurities. The process results in a high yield and purity product during crystallization step. Moreover, the recycling of mother liquor like done in usual crystallization processes mentioned in some of prior art is not necessary. Thus the overall process is simple, economical, scalable and does not need the additional steps for purification of said chlorinated sucrose or its derivative/s.

In the embodiment of this invention involving in situ deacylation of TGS-acetate or de-benzoylation of TGS-benzoate on the chromatographic column, the present invention provides an improved and integrated adsorptive chromatographic process for removal of tertiary amide solvent and all organic and inorganic salts, accompanied by capture and hydrolysis of hydroxyl group protected chlorinated sucrose derivatives, and their further recovery from the pH adjusted reaction mixture from chlorination reaction of sucrose or its derivatives (termed ‘chlorinated reaction mixture’ hereafter) in partially purified form that can be further purified by any or known processes. The invention relates to use of a single adsorptive chromatographic step, which comprises
e) contacting the pH adjusted chlorinated reaction mass with a pre-equilibrated adsorbent matrix whereby the 6-O position hydroxyl group protected chlorinated sucrose derivatives, and other chlorinated derivatives of sucrose adsorb onto the adsorbent, and
f) washing the adsorbent matrix to remove any unadsorbed compounds including DMF and all salts, and
g) simultaneous hydrolysis and progressive and/or selective desorption of the chlorinated sucrose derivatives to recover de-protected chlorinated sucrose derivatives, including TGS using an appropriately formulated regeneration/elution mobile phase, and
h) flushing and equilibrating the adsorbent matrix for reuse.

The invented process is a novel process that performs multiple steps in one equipment which can be a batch contactor (such as stirred tank), a packed bed chromatographic column, or expanded bed column, fluidized bed column, liquid solid circulating fluidized bed (LSCFB), moving bed, or a membrane chromatographic system (such as hollow fiber, spiral, or sheet), or a centrifugal chromatographic system, or any combination thereof. The combination of these equipment can be such as expanded bed and packed bed, fluidized bed and packed bed and so on, and which gives enhanced performance of the process of invention.

The process is also useful for de-protection of hydroxyl groups other than 6-O-protected group of chlorinated or non-chlorinated sucrose derivatives. Such protection of hydroxyl group can be at one or more than one hydroxyl moiety, for example diester, triester, tetraester or pentaester.

An embodiment of the process of this invention removes the water from the aqueous or aqueous-organic solution of purified or partially purified chlorinated sucrose derivative/s below 5% v/v moisture level. Further, the process also performs concentration of the product to more than 5% w/v concentration from dilute solution of chlorinated sucrose derivative/s such as TGS, and obtains the solution in organic solvent/s. The solvent, or combination of solvents, used in the present process are mostly, but not necessarily, those which form the azeotrope with water so as to aid in complete removal of residual water during distillation or evaporation. The solvents used are such that their azeotropes with water are low temperature boiling, and can be quickly distilled.

Crystallization of the concentrated chlorinated sucrose derivative/s is then carried out to isolate more than 90% of product having HPLC purity of more than 99%. The crystallization is carried out using one or combination of solvent/s in which chlorinated sucrose derivative/s have low or partial solubility.

In this embodiment, the process of the present invention comprises capture, water removal and concentration of an aqueous or aqueous-organic solution of purified or partially purified chlorinated sucrose derivative/s by chromatography using porous adsorbent matrix wherein,
i) the solution containing purified, or partially purified, chlorinated sucrose derivative/s is brought in contact with a pre-equilibrated non-ionic, ionic or mixed mode adsorbent matrix whereby the said chlorinated derivative/s adsorbs onto it, and
j) draining and/or purging the absorbed matrix using air or an non-reacting gas to remove free held water, or water containing solvent, in the settled adsorbent bed, and
k) elution of the said chlorinated sucrose derivative from the matrix using a substantially water free solvent, or mixture of such solvents, and
l) regenerating and re-equilibrating the adsorbent matrix for reuse in the next cycle.

After adsorption of the chlorinated sucrose derivatives, including TGS, the adsorbent matrix, preferably used in a packed column, is settled, drained and purged with a non-reacting gas such as air, nitrogen so as remove the free water, or water containing solvent, held up in the settled adsorbent bed void space. The adsorbed mass is then eluted from the adsorbent matrix in a suitable single solvent, or a mixture of solvents, as a concentrated mass with moisture content less than 5% v/v as analyzed by Karl fisher method. Thus, the method of the present invention performs water removal and concentration in single step. The concentrated eluted or desorbed solution is then subjected to distillation or evaporation under vacuum at 30 to 60 degree Celsius and crystallized from the solvent. The developed process results in increased yields after crystallization due to concentration and complete removal of water.

None of the prior art discloses the capture, water removal and concentration by any type of adsorption chromatography. Also none of prior art has higher crystallization yields for desired chlorinated sucrose derivative/s than those obtained by the present invention.

### BRIEF DESCRIPTION OF FIGURES

FIG. 1. The FIGURE showing performance comparison of purification trials using SEPABEADS SP700 (Mitsubishi Chemical Corporation, Japan), for the process of the present invention in terms of matrix capacity, (gm/liter), TGS recovery, (%), and DMF recovery (%) as per Example 6.

### DETAILED DESCRIPTION OF THE INVENTION

The typical reaction mixture for preparation of TGS, in addition to the protected and/or deprotected TGS or related moieties also contains mono, di, tri, and tetra chloro derivatives of sucrose, dimer or mulitmeric impurities, high boiling solvents, and salts like chlorides, phosphates, acetates, benzoates generated during neutralization and hydrolysis after chlorination step. In particular, all these impurities present a complex downstream processing problem and can seriously affect the economics of the TGS manufacturing process. Reaction mixture to be subjected to the column chromatographic process of this invention may also be a result of enzymatic acylation of sucrose further subjected to enzymatic deacylation or a neutralized chlorination reaction mixture subjected to enzymatic deacylation. In both the cases, any process step involving isolation and concentration of TGS-6-acetate is, consequently, redundant and the process of this invention may be considered omitting the step of isolation of TGS-6-acetate or TGS-6-banzoate and its purification or deacylation. When present, presence of various mono, di-, tri-, and tetra chloro sucrose derivatives interfere with the formation of pure TGS, and thereby decreases the yield and purity of TGS, These are only partially removed during conventional purification processes and interact with the flavor systems of food and beverage products in adverse ways on a account of their varying degrees of sweetness and a profound adverse effect on taste and the quality of end product. Conversely, the removal of all impurities may beneficially affect taste, sweetness, and palatability. Also the solvent used in the synthesis route e.g tertiary amide, often affects the crystallization of product, and needs to be removed. This solvent removal has been found difficult to remove by known conventional processes such as distillation or steam stripping. Thus there are multiple problems during the downstream processing of a reaction mixture containing TGS.

These complex problems are solved in this invention by the embodiments of this invention which include using adsorbents, to be used in column chromatography, which have some degree of affinity fairly specific to one or more of a desired chemical molecule, including but not limited to DMF, chlorinated sucrose derivatives, or chlorinated sucrose, intended to be removed under operating conditions. This said relative affinity is more selective than adsorption-desorption of molecules generated in prior art column chromatographic processes comprising hydrophobic:hydrophilic interactions with ion exchange or silica gel adsorbents, and results in selective chromatographic retention behaviour generated between adsorbent, molecular species to be separated and the eluant.

For the purpose of this specification, affinity chromatography comprises use of an adsorbent surface that can display a degree of relative affinity for the target molecule over some or all of the other components of the mixture.

For the first time in chromatographic separation of TGS, TGS precursors, and TGS derivatives the process of this invention has used such adsorbents, which have widely differing affinities with respect to the closely similar molecules encountered in the process for production of chlorinated sucrose to make it possible to achieve their separation without overlap in column chromatography

This feature of this invention has not only made the process of column chromatography highly efficient but has also opened up possibility for the first time of integrating in situ deacylation of adsorbed TGS-acetate or de-arylation of TGS-arylate (deacylation of TGS-6-acetate or TGS-6-benzoate, while it is still inside the column either in contact with the adsorbent or in a state desorbing from the adsorbent) by alkaline aqueous eluants while it is in the process of desorption, which is one of the embodiments of this invention. Very important advantage of in situ deacylation is that it proceeds in absence of DMF, which avoids DMF being exposed to alkaline conditions, and is recovered practically in toto without destruction, which has significant economic advantage over earlier prior art processes involving deacylation of TGS-acetate or de-arylation of TGS-6-arylate in presence of DMF in a liquid reaction mixture. Further, there is simultaneous isolation of TGS-acetate or TGS-benzoate from all impurities in the mixture and elution in a significantly pure form, all integrated in one and the same process step of affinity column chromatography. The process thus performs several multiple functions in single step. The recovered deprotected chlorinated sucrose derivatives can be further purified by any or known processes like chromatography and/or extraction followed by crystallization. This method of deacylation is not anticipated in any of the prior art and has led to development of a very simple and highly economic process of production of TGS in particular and halogenated sugars in general.

The process can recover the de-protected chlorinated sucrose derivatives in concentrations higher than concentrations of the compounds in the reaction mixture used as input to the invented process.

A further embodiment of this invention serves as a very effective process for concentration and makes it possible to process large volumes of dilute solutions which are converted into solutions of 5% w/v or more concentration of desired chlorinated sucrose product of high purity, free from all other impurities including DMF. A concentration up to a final content of 5% v/v or less has also been achieved all in one single process starting from dilute complex reaction mixtures, with about 95% or more of the recovery of desired product, substantially free from all impurities, in a single pass, and the isolated desired product can further be made free from traces of impurities by just one more pass through another column having same or different adsorbent.

In a further embodiment of this invention, the adsorbent can be regenerated repeatedly for a large number of times. The recovered product does not need any other methods for further purification. Thus overall process is simple, economical and scalable. Throughout this specification, unless the context doesn't permit or indicates to the contrary, a singular includes pleural of that kind also e.g. “an affinity chromatographic process” includes one or more of chromatographic processes based on affinity chromatography. Similarly, “a solvent” includes one or more solvents. “a process stream” for production, purification and isolation of TGS, TGS precursors and TGS derivatives includes one or more or all of the “process streams” encountered in process steps of all known processes for production, purification and isolation of TGS, TGS precursors and TGS derivatives.

The embodiments of reaction mixture/process stream/process solutions to which this invention is applicable includes those all from a simple solution of TGS-acetate or TGS made in water from which the respective solutes are intended to be recovered again, to any process stream derived from a process, enzymatic as well as non-enzymatic, of production of TGS-acetate or TGS, which includes but not limited to, one or more of TGS, TGS precursors and TGS derivatives.

In the present invention, an entirely novel approach is taken. The neutralized reaction mass, which comprises of the mixture of chlorinated sucrose derivatives either in 6-O-protected or de-protected or mixtures thereof is subjected to contact with a suitable ligand (adsorbing agent) which has specific affinity with the target product present in the mixture to be separated. This ligand could comprise of probable adsorbent derived from cross-linked polystyrene-divinylbenzene or polymethacrylate based matrices or derivative made there from by suitable surface modification which will selectively adsorb the chlorinated sucrose derivatives on to it. The inorganic salts, solvent and water are then separated from the said ligand as liquid. The adsorption could be selective for one sucrose derivative or more than one sucrose derivative could be adsorbed on to the column matrix which then could be selectively desorbed by appropriate eluants.

The interaction between the adsorbing agent and the chlorinated sucrose derivatives could be based on the formation of a temporary bond between the said adsorbing agent and the sucrose derivative.

In the present invention includes, without limiting the invention to, identifying one or more of a suitable ligand for the separation of sugar derivatives to accomplish the temporary bond formation between the ligand and the sugar derivatives, which is an improvement over any of the other prior art processes. The separation in such a case is based on pure hydrophobic affinity and not on polar interaction.

The ligand as described shall adsorb chlorinated sucrose derivatives and shall separate out and help to wash away all other constituents of the neutralized reaction mass. The chlorinated sucrose derivatives shall then be extracted from the adsorbent in a progressive way from the first chlorinated sucrose followed by the second chlorinated positions and so on, as appropriate.

Each of the fractions from the selective desorption shall be collected separately. Then the fractions will be concentrated, de-protected and crystallized by conventional methods.

Illustrative list of the said embodiments of a reaction mixture/a process stream/a solution which can be purified by process of this invention more specifically includes for the purpose of more specific illustration, without being limited to, solutions containing one or more of a chlorinated sucrose, derived from a process stream of one or more of a process of production of TGS including one or more of the following:
a) Isolation and concentration of the organic sucrose derivatives free from DMF and/or inorganics and/or from other organic impurities including degradation products from enzymatic as well as non-enzymatic reaction mixtures including the chlorination reaction mixture and from plain solutions of TGS and/or TGS-acetate and/or TGS-arylate such as TGS-benzoate in an aqueous or non-aqueous solvent;
b) Removal of inorganics and organic impurities from the solids obtained by drying the reaction mixture by various methods of drying including ATFD (Agitated Thin Film Dryer as described in Ratnam et. al. WO/2005/090374 and Ratnam et al WO/2005/090376), after dissolution in aqueous or non-aqueous medium;
c) Concentration of product fractions obtained after purification from column chromatography or other purification methods;
d) Separation of glucose-6-acetate from sucrose-6-acetate in an enzymatic conversion process.

Many more embodiments of process streams to purification of which the process of this invention can be applied thus can from several prior art processes, enzymatic as well as non-enzymatic, of production of TGS, of production of precursors of TGS and of production of derivatives of TGS. Such processes include, without being restricted to, Fairclough, Hough and Richardson, Carbohydrate Research 40 (1975) 285-298, Mufti et al (1983) U.S. Pat. No. 4,380,476, Rathbone et al (1986) U.S. Pat. No. 4,380,476, O'Brien et al (1988) U.S. Pat. No. 4,783,526, Tully et al (1989) U.S. Pat. No. 4,801,700, Rathbone et al (1989) U.S. Pat. No. 4,826,962, Simpson (1989) U.S. Pat. No. 4,889,928, Navia (1990) U.S. Pat. No. 4,950,746, Horner et al (1990) U.S. Pat. No. 4,977,254, Walkup et al (1990) U.S. Pat. No. 4,980,463, Neiditch et al (1991) U.S. Pat. No. 5,023,329, Vernon et al (1991) U.S. Pat. No. 5,034,551, Walkup et al (1992) U.S. Pat. No. 5,089,608, Dordick et al (1992) U.S. Pat. No. 5,128,248, Khan et al (1992) U.S. Pat. No. 5,136,031, Bornemann et al (1992) U.S. Pat. No. 5,141,860, Dordick et al (1993) U.S. Pat. No. 5,270,460, Navia et al (1994) U.S. Pat. No. 5,298,611, Khan et al (1995) U.S. Pat. No. 5,440,026, Palmer et al (1995) U.S. Pat. No. 5,445,951, Sankey (1995) U.S. Pat. No. 5,449,772, Sankey et al (1995) U.S. Pat. No. 5,470,969, Navia et al (1996) U.S. Pat. No. 5,498,709, Navia et al (1996) U.S. Pat. No. 5,530,106, Catani et al (2003) US patent application no. 20030171574, Ratnam et al (2005) WO/2005/090374, Ratnam et al (2005) WO/2005/090376 and the like. This is only an illustrative list, not claimed to be exhaustive and complete.

In one embodiment of this invention, the invention relates to an adsorption chromatographic process for isolation and purification of a reaction mixture/a process stream/a solution containing chlorinated derivatives of sucrose and including TGS, and to make TGS substantially free from most hydrophobic and hydrophilic impurities, inorganic and organic salts, solvents and colored residues. More particularly, the invention relates to a process of high yield and purity by which TGS can be isolated from a reaction mixture. Specifically, it relates to a process for separating TGS in pure form by which substantially pure TGS free from structurally related and non-related impurities present in the reaction mixture, can be separated and recovered in a high yield at a high recovery ratio of, for example, more than 95% and sometimes as high as 100%. This process is accomplished by the use of an apparatus, and using a process that involves adsorption, washing and elution or desorption operations without the need for any additional pre-purification while maintaining satisfactorily rates of recovery and good durability of the adsorbent, and which gives 99% pure TGS with more than 95% recovery.

The prior art patents do not cover the use of rigid porous matrices based on polystyrene divinyl benzene (PS-DVB), polymethacrylates, cellulosic matrices, porous gel type matrices based on agarose, chitosan, dextran, polyacrylamide, and matrices based on hydroxyapatite, controlled pore glass, stainless steel, quartz, magnetic beads. Also the patents do not disclose the use of expanded bed, fluidized bed, solid liquid circulating fluidized bed, membrane chromatography, packed bed, tandem column chromatography and simulated moving bed chromatography for above type of matrices. Further, the prior art patents do not disclose the particle size, pore size, surface area of the matrix required for desired purification, type of group, other than sulfonic acid group, like carboxylic, amino, diols, cyano, aliphatic, aromatic, halogen and metal chelating groups like imminodiacetic acid (IDA) for immobilized metal chelate affinity chromatography. Finally, the prior art patents do not disclose the use of modified silica such as silica with aliphatic and/or aromatic moiety (C1 to C8 carbon atoms), cyano or amino group. Also the suggested use of the chromatographic process having pulse operations may be ill suited for handling large volumes of feed material.

The prior art patents do not disclose the use of natural or synthetic polymeric matrices in column chromatography in different modes such as those mentioned above.

In addition, relatively little attention has been focused on other approaches for removing halogenated sugar impurities from TGS.

According to preceding discussion it is noted that none of the processes patented or reported, addresses the problems identified in the background of the invention. Therefore a need was felt to invent a scalable, economical and commercially viable process for purification of chlorinated derivatives of sucrose including deprotected TGS, protected TGS and partially protected TGS, and for removal of the solvent DMF without significant loss and degradation. The same method can also be used for isolation of TGS or TGS-acetate alone from their liquid solutions. Also a need was felt for a process that produces TGS of high purity and ensures high yield of TGS during the final crystallization process. The loss of TGS during crystallization can be minimized if TGS is obtained free from other chlorinated sugar derivatives and DMF. None of the patented processes gives the TGS free from all chlorinated sugars derivatives and DMF in an easy and economical manner in that they require multiple processing steps of extraction, crystallization of intermediates or TGS.

The process of the present invention has overcome the above mentioned disadvantages of all processes by capture and purification of TGS and/or protected or partially deprotected TGS from other chlorinated sucrose derivatives obtained from neutralized chlorinated reaction mass using rigid polymeric adsorbent matrices in an adsorption chromatographic process. The process of present invention also removes a tertiary amide solvent such as DMF during capture of above components while salts and most of the colored residues are also removed simultaneously. Thus the process of present invention is an integrated process for capture, purification of protected and/or deprotected TGS, and removal of salts and DMF directly from reaction mass. Further, the solution of above mentioned problem is described in detail as follows.

TGS is prepared from sucrose by first protecting the most reactive hydroxyl group at the 6^{th} position of sucrose and then subjecting the 6-O-protected sucrose to chlorination using the “Vilsmeier-Haack reagent”. The chlorinated reaction mass is then neutralized with a suitable base. The constituents of the neutralized mass are as follows
a) Chlorinated sucrose derivatives (Either in 6-O— protected or de-protected and or mixture of protected and de-protected); and or
b) Inorganic salts (Phosphates, chlorides, etc); and or
c) Organic salts (acetates, benzoates, etc); and or
d) Tertiary amide, alcohol, pyridine etc. as solvent; and or
e) Colored sugar derivatives such as caramelized sugars; and or
f) Water

This neutralized mass after chlorination is processed for purification of TGS and/or protected or partially deprotected TGS from other chlorinated derivatives.

In the present invention, the neutralized reaction mass, which comprises of the mixture of chlorinated sucrose derivatives either in 6-O-protected or de-protected (chemically or enzymatically) or mixtures thereof is subjected to contact with a suitable rigid porous polymeric matrix. The matrix surface, the ligand or chemical group on the matrix has affinity and/or strong interacting ability for chlorinated sucrose derivatives, and thus can be made, under suitable conditions, to selectively adsorb chlorinated sucrose derivatives on to it. The salts, solvent and most of the colored residues are then separated from the said matrix as unabsorbed portion. The adsorption could be for one or more than one sucrose derivatives. The degree of adsorption of different chlorinated sugars differs accordingly. The degree of adsorption or binding strength or affinity from the most adsorbed chlorinated sugar to least adsorbed chlorinated sugar is tetrachloro>trichloro>dichloro>monochloro derivatives or tetrachloro<trichloro<dichloro<monochloro derivative depending upon process conditions. The interaction between the porous adsorbing matrix and the chlorinated and/or non-chlorinated sucrose derivatives is based on reversible multiple or multipoint and/or mixed mode interactions involving two or more type of interactions such as co-ordinate interaction, hydrogen bond, ionic interaction, dipole-dipole, induced dipole and hydrophobic interaction ultimately leading to a selective interaction with chlorinated sucrose derivatives and the interacting group.

The process of this invention for isolating and purifying of said chlorinated sucrose derivative in pure form where a non-ionic or anion exchange porous matrix was used comprises a matrix such as (A) a styrene and divinylbenzene (PSDVB) copolymer or (B) a copolymer of styrene, divinylbenzene, an unsaturated or saturated aliphatic and/or an aromatic moiety of a C1 to C18 carbon molecules, or halogen for example fluorine, bromine, chlorine and the like or (C) a natural polymer based e.g. agarose, dextran, chitosan or cellulose, or (D) Polymethacrylate or polyacrylamide copolymer prepared by cross-liking to form a beaded matrix or (E) combination thereof or (F) magnetic beads prepared from one or more than one of above polymeric materials or (G) modified silica having aliphatic and/or aromatic, amino or cyano moiety.

In the present invention the term “porous matrix” includes the microporous, macroporous, mesoporous, supermacroporous and gigaporous matrices.

In the context of present invention the term “affinity” means the relatively specific strength of interaction of a molecular species with the adsorbent, or one or more interacting groups on the adsorbent surface, that results in selectivity of adsorption and involves different forces of interaction depending upon type of adsorbent and mobile phase used.

The interacting group and/or ligand on the adsorbent matrix may be the part of base matrix or may be grafted on the matrix by any of the known activation chemistries to give the desired characteristics such as the matrix hydrophobicity or hydrophilicity, group density, its spatial orientation and a selective and specific affinity towards a sucrose derivative. The other properties of the matrix that are important are surface area, porosity, particle size, pore radius, and pore structure.

In the present invention membranes can also be used as an adsorbent where the interacting groups and/or ligand is distributed on the surface of membrane and such system is used as membrane chromatography. The membranes used can be porous or nonporous and in the form of module such as but not limited to hollow fiber, flat sheet, spiral membrane based on polyether sulfone, cellulose acetate, regenerated cellulose, nylon, polytetrafluoroethylene (PTFE) and cellulose acetate phthalate. In the preferred embodiment of present invention the cross flow type of membranes are used to avoid concentration polarization effect.

In another embodiment of the present invention, the ligand on the matrix is a halogen suitable for use in the context of the present invention and includes bromine, chlorine, fluorine, and iodine. One skilled in the art may put same halogen or with any combination or permutation of different halogens, by methods known to those skilled in the art on modifications of adsorbents.

The process of the present invention is preferably carried out but not limited to using commercially available chromatographic adsorbent media. The adsorbent matrix, commercially available or otherwise, is selected from the following groups including but not limited to:
a) a copolymer of styrene divinylbenzene with or without substituted groups such as saturated or unsaturated aromatic or aliphatic moiety of C2 to C18 carbon atoms or cyano, for example DIAION HP-20, HP-21, HP20SS, DCA 11 or SEPABEADS SP825, SP700, SP850, SP20SS, SP70, FP-OD (Mitsubishi Chemical Corporation, Japan), Biobeads SM (BioRad Laboratories, USA), Amberchrom CG 71, CG161, CG300, CG1000 SMC adsorbents (TOSHO Bioscience), Amberlite XAD series (Rohm and Haas, U.S.A.), ADS 600 (Thermax, India) and or
b) a copolymer of styrene divinylbenzene with or without substituted groups such as halogen atoms for example fluorine, bromine, chlorine and iodine, for example SEPABEADS SP207, SP207SS (Mitsubishi Chemical Corporation, Japan), and or
c) polymethacrylate copolymers and/or polyacrylamide copolymers prepared by cross liking to form a beaded matrix with or without substituted groups, for example, DIAION HP-2MG (Mitsubishi Chemical Corporation, Japan), Macro-Prep methyl, butyl, phenyl (BioRad Laboratories, USA), and or
d) a natural polymer based matrix e.g. agarose, dextran or cellulose, for example SOURCE 5 RPC, 15 RPC, Phenyl Sepharose 6 FF, HP, high substitution, Butyl and Octyl Sepharose 4FF (GE healthcare), CELBEADS (Indigenously developed, Indian patent application No. 356/Mum/2003), and or
e) modified silica with aromatic and/or aliphatic moiety or cyano as substituted group having C2 to C18 carbon atoms, and or
f) Mixed mode or anion exchange matrices based on one or more than one of above polymers and having amino (primary, secondary or tertiary) or imino moiety, for example SEPABEADS FP-NH₂, EB-QA, EB-DA, FP-HA, EB-HA (Resindion srl, Mitsubishi Chemical Corporation, Italy), Sepharose-DEAE, Streamline-DEAE (GE healthcare), CELBEADS-DEAE (Indigenously developed, Indian patent application No. 356/Mum/2003), and or
g) matrices based on PSDVB, polymethacrylates, polyacrylamide, natural polymers and combinations thereof having hydroxyl or diol group, for example SEPABEADS FP-HG (Resindion srl, Mitsubishi Chemical Corporation, Italy)

Other properties of the matrices used in process of the present invention are surface area (at least 100 m²/g), pore diameter (at least 50 Å), particle size (at least 5 μm), and solubility index or hydrophobicity (at least 0.5).

The resins used in the two-adsorption steps can be identical or different. Selection of resin is critical and needs lot of experimentation and the selection depends on properties of resin (pore size, grain size, surface area, base matrix and surface hydrophobicity, and solubility index), type of material to be purified, level and nature of impurities or related impurities present, type of medium used for reaction and mobile phases used. Other factors that play role in the selection are chromatographic conditions like temperature, flow rate, gradient or isocratic method, gradient shape and gradient volume.

In the present invention the term “related impurities” means the impurities generated during the synthesis or during processing before the chromatographic step and are structurally related to the said chlorinated sucrose derivative.

In the present invention the term “gradient elution” includes stepwise, linear, convex and concave gradient effected in the composition/properties of the mobile phase used for selective desorption/elution of TGS and other chlorinated derivatives of sucrose. The term “gradient volume” means the volume of mobile phase in which the final strength of eluting mobile phase is achieved.

The adsorption capacity of the adsorbent matrix when contacted with neutralized reaction mass is between 5 and 100 gm/lit for deprotected TGS, for protected TGS and for partially deprotected TGS (i.e. mixture of protected and deprotected TGS) individually or combined. The process can be carried out in batch or in continuous mode. According to one embodiment, the adsorption is preferably performed with a packed bed chromatographic column, which comprises filling the column with a suitable adsorbent and passing the reaction mass through the resin column.

For the continuous mode of operation packed bed or expanded bed adsorption (EBA) or fluidized bed adsorption (FBA) or liquid solid circulating fluidized bed (LSCFB) or membrane adsorption (MBA) or improved simulated moving bed (ISMB) or moving bed or any combination thereof can be used. In case of batch system a stirred tank or agitated tank can be used.

In the process of the present invention when expanded bed or fluidized bed is used for purification, elution after loading and washing stage can be performed in expanded, fluidized or packed bed mode. Preferably the elution is carried out in packed bed mode.

According to the preferred embodiment, the said chlorinated sucrose derivatives are adsorbed onto the matrix or membrane which are then washed to remove salts and DMF followed by selective elution to obtain pure protected, deprotected or partially deprotected TGS. The said TGS may not only be purified by the stepwise or linear gradient of mobile phases but also by the isocratic elution with a suitable mobile phase.

In the preferred embodiment of the present invention where the ligand or interacting chemical group on the matrix or membrane is benzyl or phenyl group on the matrix, the trichloro and tetrachloro sucrose derivatives are retained whereas dichloro and monochloro derivatives are isolated in wash mobile phase. The trichloro and tetrachloro derivatives are then isolated in pure fractions by selective elution.

In another preferred embodiment where, if the ligand or interacting group on the matrix or membrane is an aromatic halogen such as bromine, then all tetrachloro, trichloro, dichloro and monochloro derivatives are found to adsorb. The binding strength of these can be in the order of tetrachloro>trichloro>dichloro>monochloro derivatives or tetrachloro<trichloro<dichloro<monochloro derivatives under alkaline and acidic conditions respectively. The desired chlorinated sucrose derivative e.g. TGS, is then selectively eluted after washing of either dichloro or monochloro in former case, and tertachloro in latter case. Similarly, when the mixture of trichloro, dichloro and monochloro sucrose derivative are subjected to another type of ligand such as amino, or imino, the trichloro sucrose derivatives are found to adsorb strongly whereas the dichloro and monochloro derivatives can be eluted out selectively. After this the tichloro derivative can be eluted as pure fraction. Thus, by the present invention the reaction mixture containing monochloro, dichloro, trichloro and tetrachloro derivatives and/or mixture thereof can be separated into pure fractions in several different routes and their combinations. The DMF and salts remained unabsorbed in any of above route and can be simply washed from the adsorbent without loss of the adsorbed chlorinated sucrose derivatives.

In the process of the present invention when the reaction mass containing deprotected TGS is used as feed, after elution more than 95% pure form of deprotected TGS is obtained.

In the process of the present invention when the reaction mass containing protected and/or partially deprotected TGS is used as feed, selective elution gives more than 95% pure form of protected and/or partially deprotected TGS. This protected and/or partially deprotected TGS is then completely deprotected after purification by known conventional chemical or by enzymatic methods.

In an embodiment of the process of the present invention the purified deprotected TGS is finally polished in a second chromatographic column on a similar or another type of adsorbent, after complete or partial removal of the organic solvent used in the eluting mobile phase by simple evaporation or distillation. The adsorbent used in the polishing step can be selected from the entire group of adsorbents mentioned above. The capacity of the matrix in polishing step is more than 5 gm product/lit of adsorbent. This polishing step can be operated in packed bed, simulated moving bed, or any improved simulated moving bed. Hydrophobic interaction chromatography, affinity chromatography, ion exchange chromatography, ion exclusion chromatography, reversed phase chromatography, membrane chromatography, centrifugal chromatography and mixed mode interaction chromatography or combination thereof can be used. Tandem column chromatography, wherein a select fraction eluting from one column can be directly fed into a second column, can also be used for such process. The isocratic or gradient elution can be used to remove the traces of the impurities so as to get the TGS of more than 99% purity. In another case of present invention the protected and/or partially deprotected TGS can also be polished by such process to get more than 98% pure form of protected and/or partially deprotected TGS which then can be hydrolyzed by known conventional or enzymatic methods to give pure TGS. The said chromatographic process after purification and polishing gives the recovery of more 90% and sometimes as high as 100% of pure TGS-6-acetate, TGS-6-benzoate or TGS with respect to the input feed with respect to feed reaction mass. Any fraction from polishing column showing trace impurity is then recycled in the next cycle to get overall recovery higher than 95%.

In yet another embodiment of the process of the present invention, the neutralized chlorinated reaction mass containing deprotected TGS or protected and/or partially deprotected chlorinated sucrose derivatives can be dried by known method such as agitated thin film drier, whereby the solvent portion gets removed. This dried reaction mass can be dissolved in aqueous or aqueous-organic medium and the pure TGS can be recovered according to the process of the present invention.

In yet another embodiment of the process of the present invention the solvent free or aqueous chlorinated reaction mass containing deprotected TGS or protected and/or partially deprotected chlorinated sucrose derivatives and process feed obtained using any type of chromatographic step can be used as feed. Further, pure TGS or chlorinated sucrose can be recovered according to the process of the present invention.

The equilibration, washing, elution and regeneration mobile phase in both purification and polishing contains the organic modifier such as but not limited to alcohols (methanol, ethanol, isopropanol, butanol), acetonitrile, chlorinated organic solvents (chloroform, diclhloromethane, dichoroethane) toluene, esters (butyl acetate, ethyl acetate), ketones (acetone, methyl isobutyl ketone), and any suitable combination of one or more than one thereof. Water may also be combined with these solvents to adjust and manipulate the desired affinity and/or interaction ability of the mono, di, tri and tetra chlorinated compounds with the adsorbent as required. Water can be also be used in proportion from 0% to 100% depending on the type of reaction mass charged on the adsorbent, and the required washing, elution, regeneration and equilibration conditions. For example, in case of equilibration 100% water was used whereas for regeneration water concentration used was as low as 0%.

The mobile phase used may also contain suitable ion-pairing agent/s and/or affinity and/or binding strength modifiers such as, but not limited to, phosphoric acid, acetic acid, pentane sulphonic acid, trifluoro acetic acid, triethylamine and any suitable combination of one or more than one thereof. The concentration of ion-pairing agent in the mobile phase ranges from 0.001% v/v to 2.5% v/v depending upon the type of ion-pairing agent selected. Buffer such as, but not limited to, citrate buffer, phosphate buffer, acetate buffer, phosphaste-citrate buffer (Macllav buffer), citrate-acetate buffer, borate buffer, carbonate buffer can be used for creating the difference between interactions or binding strength of said chlorinated sucrose derivatives with the matrix.

In the preferred embodiment of present invention food grade salts, buffers, acids and alkalis are used.

The mobile phase used for equilibration, washing, elution and regeneration is applied to the adsorbent in an unchanged manner as in ‘isocratic elution’, or step wise manner or in a changing manner over any suitable period of time and over any volume of liquid, as in ‘step gradient’ or any suitable ‘continuous gradient’ elution, or any combinations thereof.

In the course of chromatographic purification methods invented, each of the desorbed fractions obtained from the selective desorption from the adsorbent is collected separately and analyzed for TGS content by HPLC, solvent content by GC and for other chlorinated derivates by TLC according to known procedures. Then the pure fractions are combined and the concentrated. TGS obtained by the process of present invention is then crystallized by known conventional processes to get solid TGS having purity of more than 99% on weight % basis.

The advantages of the process according to the invention can be summarized as follows:
(1) In the adsorption step carried out with the neutralized native reaction mixture or dried reaction mass, the said TGS and/or protected or partially de-protected TGS is purified with simultaneous removal of all salts and solvent/s.
(2) The process is economical due to reusability of adsorbent after regeneration
(3) The process has enhanced effect on final crystallization yield and purity of said chlorinated sucrose derivative.
(4) The process gives the said chlorinated sucrose derivative, TGS in more than 99% purity and more than 95% yield.
(5) The process can be carried out from laboratory to industrial scale. Most advantageously, it can be carried out in any of the packed bed, expanded bed, fluidized bed, liquid solid circulating fluidized bed, improved simulated moving bed or by membrane chromatography modes which makes continuous operation possible.

The principle of adsorption and chromatography can be applied at various stages in the process for the isolation of the chlorinated sucrose derivatives. Some of the stages where it can be substituted or incorporated with variations included are as follows:
a) Affinity adsorption chromatography can be applied before or after de-protection of the neutralized chlorinated sucrose derivatives
b) Can be applied before or after the removal of the tertiary amide
c) Can be applied for removal of tertiary amide and salts from said chlorinated derivatives
d) Can be applied before or after the removal of salts partially or completely (organics an/or inorganics)
e) Can be applied at any stage after partial purification of chlorinated sucrose derivatives through any of the other operations such as extraction, chromatography, crystallization, distillation, etc
f) It can be used as a substitute to traditional column chromatography
g) It can be applied to purify or isolation of any sucrose intermediate compound used for the preparation of the said chlorinated sucrose derivatives.
h) It can be applied for the purpose of further purification of isolated TGS or its precursors or derivatives by subjecting the solution for column chromatography of this invention
i) Variation in pH conditions to increase or decrease adsorption or binding strength of chlorinated sucrose derivatives in the feed solution

Details of the embodiment relating to the said in situ deacylation of protected chlorinated sucrose itself includes more than one embodiments indicated above.

In one embodiment of the process of present invention on embodiment of in situ deacylation, the process may employ a feed mixture that may contain all the monochloro, dichloro, trichloro and tetra-chlorinated derivatives of sucrose.

In another embodiment of the present invention on embodiment of in situ deacylation, the TGS compound may comprise 6-O-acetyl or 6-O-benzoyl derivative of chlorinated sucrose. The types of halogenated compounds present in this feed mixture may vary according to the synthetic route used and the particular conditions of the synthesis. Halogens suitable for use in the context of the present invention include bromine, chlorine, fluorine, and iodine. One skilled in the art may readily fill the various positions with the same halogen or with any combination or permutation of different halogens by methods known to those skilled in the art.

Also in yet another embodiment of the process of present invention on embodiment of in situ deacylation, protection of hydroxyl group can be at one of more than one position to give diester, triester, tetraester or pentaester, and may comprise acetyl or benzoyl or other suitable group. The types of these ester compounds present in this feed mixture may vary according to the synthetic route used and the particular conditions of the synthesis. One skilled in the art may readily block the various positions with the same group or with any permutation and combination of different groups by methods known to those skilled in the art.

In certain embodiments of the process of this invention on embodiment of in situ deacylation, compounds included are those, other than TGS and the products of any number of processes for synthesizing TGS that are not TGS are also hydrolyzed. These includes any monochloro-, dichloro-, tetrachloro-, and pentachloro-derivative of sucrose and any other disaccharide derived from sucrose, as well as any trichloro-derivative other than TGS itself, whether present in free form or as esters form.

The present invention provides processes whereby the reaction mass is charged to a suitable equipment from the list given above in the Summary of Invention, in order to contact the compounds in the mixture with the adsorbent matrix, and whereby the compounds viz. the protected chlorinated sucrose derivatives are de-protected, fully or partially, in their adsorbed state to produce de-protected chlorinated sucrose derivatives, and are recovered by desorption. The de-protected derivatives, including TGS, are thus recovered by chromatographic procedure. In the process the reaction mixture solvents like DMF along with all salts present in feed reaction mixture are also removed during adsorption and washing cycle of the process.

The process can be carried out in batch or in continuous mode. According to one embodiment, the adsorption is mostly performed with a packed bed chromatographic column or expanded bed chromatographic column, which comprises packing the column with a suitable adsorbent and passing the reaction mass through the column. During the loading of the neutralized chlorinated reaction mass a step or gradient type loading is employed to avoid the bed instability.

The binding capacity of the adsorbent is more than 10 gm/liter for 6-O-protected chlorinated sucrose in one case of the embodiment, whereas it is more than 50 gm/liter in another case of the embodiment. In the preferred embodiment of present invention the matrices having capacity of more than 50 gm/liter are preferred. The total binding capacity for the desired chlorinated sucrose derivative is based on the surface area and the nature, condition and composition of feed material.

In the process of the present invention the term “nature of feed material” means total content of protected and/or partially de-protected chlorinated sucrose, types and composition of chlorinated sucrose such as monochloro dichloro, trichloro, and tetrachloro derivatives in neutralized reaction mass.

In the process of the present invention the term “condition and composition of feed material” means pH, conductivity, temperature and composition in terms of presence and types of inorganic and organic salts.

In the process of the present invention the adsorbent matrix may function as “catalytic resin” for the hydrolysis of 6-O-protected chlorinated sucrose derivatives. While desorbing the 6-O-protected chlorinated sucrose derivative it gets hydrolyzed to form 6-O-deprotected chlorinated sugar. The elution mobile phase used is aqueous or aqueous-organic based and has a catalytic ions which increase the rate of hydrolysis in presence of adsorbent matrix. The catalytic ions are generally, but not necessarily, the hydroxyl ions (OH⁻) with a counter ion as sodium, potassium, calcium and/or ammonium ions. The pH of mobile phase ranges from 7.5 to 12 pH units based on concentration of hydroxyl ion and type of counter ion.

In the process of present invention the adsorbent matrix itself bears these ions, or these are externally added as part of the mobile phase, in order to effect hydrolysis of 6-O-protected chlorinated or non-chlorinated sucrose derivatives. The fully or partially hydrolyzed mixture is then desorbed or eluted from the matrix in 6-O-de-protected form such as TGS and other deprotected chlorinated sugars.

In the process of the present invention the adsorbed matrix can be washed with an alkaline solution to transform the 6-O-protected form of chlorinated sucrose derivatives to 6-O-de-protected form, and then 6-O-deprotected chlorinated sucrose derivatives, including TGS, are recovered using one or mixture of aqueous and aqueous-organic desorbent solutions. The pH of the eluted or desorbed solution mass containing the partially or fully de-protected derivatives is adjusted with suitable acid/s or salt/s.

In the another approach of the present invention the adsorbed 6-O-protected chlorinated sucrose is desorbed using aqueous or combination of aqueous-organic elution phase, and the recovered eluate containing these derivatives are hydrolyzed by known or conventional methods such as chemical or enzymatic methods after adjusting the pH if required.

Generally, following pH adjusting compounds may be used: sodium, potassium, ammonium or other acceptable salts of hydroxide, carbonate, bicarbonate, acetate, phosphates, sorbate, tartarate, and mixtures thereof. A preferred pH-adjusting compound is sodium or potassium hydroxide, ammonia, and phosphate.

In the embodiment of the present invention the pH adjusted chlorinated reaction mass, wash solution and hydrolyzing elution solution is passed through one end of the column, and reaction mixture solvents such as DMF, all salts, and fractions containing chlorinated sucrose derivatives are collected at other end of the column. In another embodiment of present invention the feed is passed in upward direction, and DMF, salts are collected from top of the column. The hydrolyzing elution phase is passed in downward direction and concentrated hydrolyzed mass is collected at bottom of the column. In the process of present invention, the hydrolysis of desired 6-O-proptected chlorinated sucrose derivatives can be from zero to 100% on the basis of feed material, depending upon the hydrolyzing mobile phase composition and flow rate through the column.

In the hydrolyzing and/or desorption step, and in the washing step in the process the mobile phase can contain a catalytic agent that assists in hydrolysis or de-protection of the chlorinated sucrose derivatives, can be hydroxyl ion in the form, of but not limited to, of sodium hydroxide, ammonium hydroxide, potassium hydroxide, calcium hydroxide etc. The other hydrolyzing agents could be used in the said embodiment of present invention.

In the process of the present invention the hydrolyzing elution mobile phase desorbs the desired chlorinated sucrose in 6-O-de-protected form, TGS. During the elution from adsorbent matrix the desired chlorinated sucrose derivatives are eluted in concentrated fraction of less than 2 bed volumes of hydrolyzing elution mobile phase, the term bed volume hereby used to imply the volume of the adsorbent used in the process. The collected elution fraction shows more than 2% of TGS. This elution mass can be then distilled at low temperature under vacuum to remove the solvent/s comprising the elution mobile phase, and thus results in a TGS solution of higher concentration than in the chlorinated reaction mixture. The TGS recovered by such process is then preferably purified by chromatography to isolate more than 99% pure TGS.

In the process of the present invention the hydrolyzing elution mobile phase itself can perform the regeneration or Cleaning-In-Place (CIP) of adsorbent matrix without requirement of additional step for regeneration. This helps to reduce the overall time cycle of the process and give increased process productivity per unit adsorbent volume per hour.

The advantages of the process according to the invention can be summarized as follows:
(1) In the adsorption step carried out with the pH adjusted chlorinated reaction mixture obtained directly from the chlorination reactor, or prepared from the solution obtained by dissolving the dried chlorinated reaction mixture, the desired protected TGS and/or protected or partially de-protected TGS is captured, hydrolyzed and recovered as deprotected TGS in single column with simultaneous removal of all salts and solvents such as tertiary amide solvent.
(2) The integrated process is economical due to reusability of the porous adsorbent matrix.
(3) The process gives completely hydrolyzed chlorinated sucrose derivative, TGS in more than 95% yield.
(4) The process is scalable and is carried out on an industrial scale. Most advantageously it can be carried out using one or more units of packed bed, expanded bed, fluidized bed, liquid solid circulating fluidized bed, improved simulated moving bed, or by membrane chromatography, which makes a continuous operation possible.

The principle of this process of separation by adsorption and/or affinity chromatography, can be applied at various stages in the process for the de-protection and isolation of the de-protected chlorinated sucrose derivatives. Some of the stages where it can be substituted or incorporated with variations included are as follows:
a) Can be applied before de-protection of the neutralized chlorinated sucrose derivatives
b) Can be applied before or after the removal of the tertiary amide
c) Can be applied for removal of tertiary amide and salts from said protected chlorinated derivatives
d) Can be applied before or after the removal of salts partially or completely (organics an/or inorganics)
e) Can be applied at any stage after partial purification of protected chlorinated sucrose derivatives through any of the other operations such as extraction, chromatography, crystallization, distillation, etc.
f) Can be used as a substitute to traditional hydrolysis method
g) Can be used for hydrolysis of chlorinated or non-chlorinated sucrose derivatives which are protected at one or more than one hydroxyl group positions.

Details of another embodiment of this invention, which involves capture, concentration and crystallization of halogenated sucrose from their dilute aqueous or aqueous-organic solutions are given in the following.

The aqueous or aqueous-organic solution of purified or partially purified chlorinated sucrose derivative/s is obtained by known processes such as extraction or chromatography which comprises,
a) a halogenated such as chlorinated sucrose derivative with hydroxyl group protected and/or de-protected or partially de-protected, and
b) water, and/or
c) Organic solvent such as alcohol (e.g. Methanol, isopropyl alcohol, ethanol etc.)

In one embodiment of the present invention, the halogenated compound/s present in this feed mixture may vary according to the synthetic route used and the particular conditions of the synthesis. Halogens suitable for use in the context of the present invention include bromine, chlorine, fluorine, and iodine. One skilled in the art may readily fill the various positions with the same halogen or with any combination or permutation of different halogens by methods known to those skilled in the art. This type of feed material can also be handled by process of present invention.

In another embodiment of the present invention the hydroxyl group de-protected or protected halogenated sucrose derivative/s is monochloro, dichloro, trichloro or tetrachloro derivative of sucrose or mixture thereof is present in the feed vehicle.

In a typical use of the process of the present invention the adsorbent matrix was loaded to more than 30 gm chlorinated sucrose/liter adsorbent using simple column chromatographic apparatus such as a cylindrical column packed with the adsorbent also called a packed bed. Although possible to operate the process in batch stirred reactor mode, it was found that a packed bed operation resulted in eluted solutions of higher concentrations of the chlorinated sucrose derivatives compared to the typical equilibrium limited batch process. The process can also be operated in other adsorbent bed modes such as expanded bed, fluidized bed, liquid solid circulating fluidized bed (LSCFB), moving bed, simulated moving bed (SMB), improved simulated moving bed (ISMB), centrifugal chromatography and annular chromatography.

Thus, in the typical process, the aqueous or aqueous-organic solution containing chlorinated sucrose derivative/s was charged to the column packed with adsorbent matrix whereby the said chlorinated sucrose adsorbed on the matrix. The loading stage was followed by draining the column under gravity, or using a suitable drive such as pump or pressurized gas, followed by purging with gas to remove almost all free water, or water-solvent mixture, held up in the matrix bed. The gas used for purging was either nitrogen or air, or mixture thereof. Desorption of adsorbed sucrose derivatives, including TGS, was carried out using a solvent, or mixture of solvents. The used solvent was selected from the group of solvents such as but not limited to alcohols (methanol, ethanol, isopropanol, butanol), acetonitrile, chlorinated organic solvents (chloroform, diclhloromethane, dichoroethane) toluene, esters (butyl acetate, ethyl acetate), ketones (acetone, methyl isobutyl ketone), and any suitable combination of one or more than one thereof.

In the process of the present invention mobile phase modifiers such as but not limited to acids (for example, phosphoric acid, acetic acid, hydrochloric acid, sulphuric acid, pentane sulphonic acid, trifluoro acetic acid, butyric acid and/or bases (for example, sodium hydroxide potassium hydroxide, ammonium hydroxide) and any suitable combination of one or more than one thereof can be used.

In the preferred embodiment of present invention food grade salts, acids, alkalis are preferably used.

The process of this invention is carried out in the range of 0 to 80 degree Celsius, preferably at prevalent ambient temperature for cost considerations.

The adsorbed chlorinated sucrose derivative/s is eluted in less than 2.0 bed volume or preferably in less than 1.5 bed volume of solvent based elution mobile phase, the term ‘bed volume’ here used to imply volume of the settled adsorbent matrix in the column, or vessel. The eluted fraction has the desired chlorinated sucrose derivative/s such as TGS in a concentration of more than 5% w/v, and contained less than 5% v/v moisture as analyzed by HPLC and Karl fisher method, respectively. The recovery on the basis of feed content of said chlorinated derivative is more than 90% and some times as high as 100%.

Further, the crystallization of concentrated mass is carried out after distillation/evaporation of elution mobile phase solvent/s or could be coupled with distillation/evaporation so as recover more than 90% of desired chlorinated sucrose derivative/s. In one embodiment of the process, a solvent in which the said chlorinated sucrose derivative/s is soluble or partially soluble, can be added to the distilled/evaporated product mass. In another embodiment, a combination or mixture of solvent/s is used in appropriate proportion to recover pure chlorinated sucrose derivative/s. In the process of the present invention a mixture or combination of solvent/s can be used for crystallization, one of the solvent being such that the desired chlorinated sucrose derivative/s such as TGS is completely or partially soluble, and the another solvent having low or very low solubility for desired chlorinated sucrose derivative/s. Such solvents can be selected from, but not necessarily limited to, chlorinated solvents (for example, methylene dichloride, chloroform, ethylene dichloride etc.), esters (for example, ethyl acetate, and butyl acetate), alcohols (for example, methanol, ethanol, butanol, isopropanol etc.), ketones (for example, acetone, methyl isobutyl ketone, methyl ethyl ketone etc.).

Further, the concentrated mass obtained from adsorptive chromatographic process can be distilled/evaporated, and finally the chlorinated sucrose derivative/s can be crystallized from the solvent by known procedure/s.

The advantages of the process according to the invention can be summarized as follows:
a) The process is highly suitable for water removal and concentration of temperature sensitive products.
b) The process of present invention does not require distillation of large volumes of water, and is thus energy efficient.
c) The process is economical due to reusability of adsorbent after regeneration.
d) The process has enhanced effect on yield and purity of said chlorinated sucrose derivative/s obtained after crystallization of such material.
e) The process gives the said chlorinated sucrose derivative/s TGS in more than 90% yield and in 99% purity.
f) The process can also integrate in it one or more of a chemical modification involving chlorinated sucrose or their compounds depending upon use of eluants suitable for the intended chemical change, including, but not limited to deacylation inside column while a process of separation by chromatography is in progress.
g) The process is scalable and can be easily adapted to industrial scale in simple packed bed chromatographic column, or other column type contactors.

This type of operation for water removal and concentration can also be applied at any of the intermediate stages during isolation and purification of said chlorinated sucrose derivative/s or their intermediates. Some of the stages where it can be substituted or incorporated with variations are as follows:
a) Can be applied before or after de-protection of the chlorinated sucrose derivative/s
b) Can be applied after enzymatic or chemical de-protection of the purified or partially purified chlorinated sucrose derivative/s.
c) Can be applied before or after the removal of the tertiary amide solvent.
d) Can be applied at any stage after partial purification of chlorinated sucrose derivative/s through any of the other operations such as extraction, chromatography, etc.
e) Can be used as a substitute to traditional extraction and distillation to remove water.
f) Can be applied after purification or isolation of any chlorinated sucrose intermediate compound used for the preparation of the desired chlorinated sucrose derivative/s.

The invention with all its major embodiments is further illustrated by the following working non-limiting examples. The examples given are mainly for the purpose of illustration and not in any way to limit the scope of the invention to the reactants, reaction conditions, adsorbents, chemicals used for the examples. Any modification or adaptation of the disclosed invention, which is obvious to a person ordinarily skilled in the art is covered within the scope of this invention. It also needs to be mentioned here that in the course of work, commercially available adsorbents have been used, however, the invention is not limited to the names of the brands mentioned, but covers the properties of that specific brand mentioned which further covers any reasonable variant of adsorbent which shall serve to represent the same or similar properties to the said brand/brands.

Any mention of a singular is construed to include its pleural too, unless not permitted by the context. Thus, mention of “a process” includes “processes” too i.e. includes all the processes covering the subject matter to which that word is directed to. A mention in singular is also construed to include all the equivalents included in that kind of matter. Thus “a solvent” includes all the solvents, which can be used to achieve the function stipulated by the claim or description.

### Example 1

### Capture, Purification and Tertiary Amide Removal

2.0 liter of neutralized reaction mass containing 20 g of TGS and other chlorinated sucrose derivatives, with inorganic and organic salts and 0.480 kg of tertiary amide was taken for purification and tertiary amide removal experiment The feed was passed through the borosilicate glass adsorption column fitted with stainless steel adaptors and filled with pre-equilibrated 0.40 L of SEPABEADS SP825 (Mitsubishi Chemical Corporation, Japan). The feed was charged using a pump at rate of 1.50 liters/hour followed by washing with deionized water. The adsorbed TGS was then selectively eluted from adsorbent matrix using 1.0 liter of 5.0% aqueous solution of isopropyl alcohol. All unbound, wash and elution fraction was analyzed for TGS and tertiary amide by HPLC and GC, respectively. The unbound and wash fraction does not show any TGS on HPLC indicating 100% adsorption efficiency of adsorbent for TGS from reaction mass. The GC result shows total 0.478 kg of tertiary amide in unabsorbed fractions. The elution fraction of 0.63 liter shows total 19.72 gm of TGS having purity of 95.30% on HPLC. The GC results show absence of tertiary amide in elution fraction. The yield related to the TGS and tertiary amide content of the starting reaction mass amounts to 98.60% in elution and 99.58% in unadsorbed fraction respectively.

### Example 2

### Capture, Purification and DMF Removal

910 liter of neutralized reaction mass containing 5.40 kg of TGS and other chlorinated sucrose derivatives, with inorganic and organic salts and 110 Kg of DMF was fed to the 180 liter SEPABEADS SP700 (Mitsubishi Chemical Corporation, Japan) packed in stainless steel column to get 2.4 meter bed height. The feed was charged using dosing pump at rate of 8.3 liter per minute followed by washing with deionized water. The adsorbed TGS was then selectively eluted from adsorbent matrix using 900 liter of 25.0% aqueous solution of methanol in water. The total 650 liter of elution fraction showing TGS on TLC was collected. The unbound and wash fraction analyzed by HPLC shows 0.080 kg of TGS indicating 98.5% adsorption efficiency of adsorbent for TGS from reaction mass. The TLC analysis of unbound fraction does not show presence of monochloro, dichloro, trichloro and tetrachloro derivatives whereas the TLC analysis of wash fractions shows presence of monochloro and dichloro derivatives of sucrose. This indicates that matrix has less affinity for monochloro and dichloro derivatives than trichloro and tetrachloro derivatives. The GC result shows total 109.2 kg of DMF in unabsorbed fractions. The elution fraction of 650 liter shows total 5.30 kg of TGS having purity of 96.80% on HPLC. The GC results show absence of tertiary amide in elution fraction. The yield related to the TGS and DMF content of the starting reaction mass amounts to 98.15% in elution and 99.27% in unadsorbed fraction.

### Example 3

### Polishing of TGS Stream

The elution fraction obtained from experiment as per Example 2 shows trace presence of dichloro and monochloro derivative of sucrose, which is removed in polishing step. Here the 13.32 kg of isolated TGS in an experiment such as described in Example 2 and after methanol removal by distillation, was charged to 500 liter of SEPABEADS SP207 (Mitsubishi Chemical Corporation, Japan) resin column having 3.98 meter bed height at 6.5 liter per minute rate. All the TGS and remaining monochloro and dichloro derivatives get adsorbed to the matrix. The adsorbed chlorinated sucrose derivatives were then isocratically eluted using 35% methanol in water. The elution fraction of 210 liter shows concentrated monochloro and dichloro derivatives and no TGS on TLC analysis. Further 1600 liter of elution fraction has 12.97 Kg of TGS without any other chlorinated derivative on TLC analysis. Total 97.52% of TGS was recovered which has purity of 99.39% on HPLC analysis.

### Example 4

### Expanded Bed and Fluidized Bed Chromatography for Capture, Purification and DMF Removal from Reaction Mass

The process was carried out in expanded bed and fluidized bed mode using 1.0 liter of SEPABEADS SP700, or SEPABEADS SP207 (both from Mitsubishi chemical corporation, Japan), or XAD 16 (Rohm and Haas, U.S.A.), or ADS 600 (Thermax, India) in 5.0 cm diameter glass column. The adsorbent was charged with 5.0 liter of neutralized reaction mass containing 60 gm of TGS-6-acetate and other chlorinated sucrose derivatives, with inorganic and organic salts and 1.2 Kg of tertiary amide solvent in each case of adsorbent. Degree of expansion used in case of expanded bed was 1.4 whereas in case of fluidized bed it was 2 times to that of packed bed height. The washing was performed in upflow mode. The adsorbed chlorinated sucrose derivatives were then isocratically eluted in downflow mode. The results of which are summarized in following Table-1 (TGS-6-acetate was analyzed as TGS after hydrolysis).

TABLE 1

| Sr. | | Expanded bed process | | | | Fluidized bed process | | | |
|---|---|---|---|---|---|---|---|---|---|
| No. | Description | SP700 | SP207 | XAD16 | ADS600 | SP700 | SP207 | XAD16 | ADS600 |
| 1 | TGS | 59.6 | 59.8 | 56.0 | 56.3 | 58.2 | 59.5 | 56.2 | 56.4 |
| | adsorbed | | | | | | | | |
| | (gm) | | | | | | | | |
| 2 | Tertiary | 1.2 | 1.2 | 1.18 | 1.68 | 1.2 | 1.2 | 1.17 | 1.64 |
| | amide in | | | | | | | | |
| | unbound | | | | | | | | |
| | and wash | | | | | | | | |
| | (Kg) | | | | | | | | |
| 3 | Adsorption | 99.3 | 99.7 | 93.3 | 93.8 | 97.0 | 99.2 | 93.6 | 94.0 |
| | efficiency | | | | | | | | |
| | with respect | | | | | | | | |
| | to TGS (%) | | | | | | | | |
| 4 | Total TGS | 59.4 | 58.2 | 54.0 | 52.5 | 58.0 | 56.3 | 45.2 | 49.4 |
| | recovered | | | | | | | | |
| | (gm) | | | | | | | | |
| 5 | Total TGS | 99.0 | 97.0 | 90 | 87.5 | 96.6 | 93.8 | 75.3 | 82.3 |
| | recovery | | | | | | | | |
| | (%) | | | | | | | | |
| 6 | Purity of | 95.3 | 96.8 | 92.2 | 90.1 | 98.2 | 97.5 | 91.4 | 89.2 |
| | TGS | | | | | | | | |
| | recovered | | | | | | | | |
| | before | | | | | | | | |
| | polishing | | | | | | | | |
| | (%) | | | | | | | | |
| 7 | Tertiary | Nill | Nill | 0.0031 | Nill | Nill | Nill | 0.0022 | 0.0025 |
| | amide in | | | | | | | | |
| | elution | | | | | | | | |
| | fraction of | | | | | | | | |
| | TGS (%) | | | | | | | | |

### Example 5

### Moving Bed Chromatography for Capture, Purification and DMF Removal from Reaction Mass

The said chlorinated sucrose derivative such as TGS (deprotected, protected or partially deprotected) was purified on liquid solid moving bed using 1.0 liter of SEPABEADS SP70 or SEPABEADS SP700 (Mitsubishi Chemical Corporation, Japan). A moving bed chromatography system operates with the resin moving down the column as the feed stream moves in upward direction as counter-current flow. The resin with adsorbed solutes is taken in another parallel column and the product eluted continuously in co-current or counter-current manner. In the present example, the reaction mixture containing said chlorinated sucrose derivatives and tertiary amide solvent as DMF was continuously fed to the main adsorption column of 5.0 cm diameter. The product was continuously eluted in the second parallel co-current column of 1.5 cm diameter after washing in bottom section of the main column. The eluted adsorbent is recycled into the top of the main column via a solid-liquid separator. The DMF and salts are continuously taken out from the top outlet of main column The system has high adsorption efficiency and small height of adsorption zone due to countercurrent adsorption in first main column.

### Example 6

### Purification of Trichloro Sucrose Derivative and Polishing

6.0 L of neutralized reaction mass containing 60 g of TGS and other chlorinated sucrose derivatives, with inorganic salts and 2.0 kg of tertiary amide was passed through 1.5 liter DIAION HP2MG (Mitsubishi Chemical Corporation, Japan) at 1.5 liter/hour rate. All the chlorinated sucrose derivatives got adsorbed to the ligand on the matrix whereas the unabsorbed fraction contained the DMF and inorganic salts. Total 1.98 Kg of DMF was collected in these fractions. Then the adsorbed matrix was washed with 2-bed volume of demineralized water to wash out any adhering DMF and inorganics. Then the Dichloro and Trichloro derivatives of sucrose are eluted using 5.6 bed volume of 30% methanol in water. The Tetrachloro sucrose derivatives remain bound to the ligand. The eluted fractions were taken for distillation to remove methanol at 40-60° C. under vacuum. Then the fraction containing Dichloro and Trichloro derivatives were passed through the polishing column containing 2.0 liter of SEPABEADS SP207SS (Mitsubishi Chemical Corporation, Japan) for further purification. Here, the dichloro derivatives were separated in the first fractions when eluted with 40% methanol in water. The first bed volume consisted of dichloro impurities which were collected separately. The next 3 bed volumes consisted of pure trichloro derivatives. The HPLC result shows 98% of trichloro derivative was recovered having 99.6% purity. This fraction was then taken for methanol removal by distillation and further to crystallization.

### Example 7

### Polishing of TGS

The elution fraction obtained as per Example 2 which shows trace presence of dichloro and monochloro derivative of sucrose in TGS which was removed in polishing step using a DIAION HP20SS, or SEPABEADS SP207SS or SEPABEADS SP20SS column. The 200 ml of each matrix was packed in separate columns to get 2 meter bed height. 4.5 g of TGS was charged to each column followed by washing with one bed volume of alkaline water, pH 9.8 and one bed volume of neutral water, pH 7.0. The Product was then eluted by gradient method using 0 to 50% gradient methanol in water in one method and 35% isocratic solution of methanol in other method. Two fractions were collected in both cases as fraction containing impurity and pure fraction containing TGS. The recoveries and purity obtained by using these matrices were summarized in following Table 2

TABLE 2

| | | HP20SS | | SP207SS | | SP20SS | |
|---|---|---|---|---|---|---|---|
| Sr. | | Isocratic | Gradient | Isocratic | Gradient | Isocratic | Gradient |
| No. | Description | method | method | method | method | method | method |
| 1 | Pure TGS | 96.2 | 97.5 | 98.8 | 98.2 | 95.8 | 94.7 |
| | recovery | | | | | | |
| | (%) | | | | | | |
| 2 | Purity of | 98.5 | 98.8 | 99.3 | 99.2 | 94.2 | 96.8 |
| | pure TGS | | | | | | |
| | recovered | | | | | | |
| | (%) | | | | | | |
| 3 | TGS in | 3.8 | 2.5 | 1.2 | 1.8 | 4.2 | 5.3 |
| | impurity | | | | | | |
| | fraction (%) | | | | | | |

### Example 8

### Isolation and Crystallization of TGS from Aqueous Solution of TGS

1000 ml of aqueous solution containing 10 gm of pure TGS obtained after extraction was passed through the borosilicate glass adsorption column fitted with stainless steel adaptors and filled with pre-equilibrated 120 ml of SEPABEADS 700 (Mitsubishi Chemical Corporation, Japan). The feed was charged using a pump at rate of 25 ml/min followed by draining under gravity. The column was then purged with air for 15 min to remove water from voidage of the matrix bed. The adsorbed TGS was then desorbed from adsorbent matrix using 200 ml 1:1 mixture of methanol:butanol. During elution initial 0.3 bed volume of water was collected as separate fraction followed by 70 ml fraction containing 99.2% of TGS. This was analyzed by HPLC showing 14.2% concentration of TGS. The moisture was 2.8% which was analyzed by Karl fisher method. The elution fraction was then distilled to remove residual water and butanol and crystallized from mixture of methylene dichloride and ethyl acetate. The purity of the crystallized product was 99.5% on HPLC.

### Example 9

### Isolation and Crystallization of TGS from Aqueous Solution of TGS at Kilogram Scale

225 liter of SEPABEADS SP700 (Mitsubishi Chemical Corporation, Japan) was packed in stainless steel column of 310 mm diameter. The matrix was washed and equilibrated with water of pH 7.5. Thirteen kilogram of purified TGS obtained from column chromatography in aqueous solution containing 2% of methanol and 98% of water was charged to the column at rate of 6 liter per min. The loading was followed by draining the column under gravity at rate of 6.0 liter per min and then air was purged through the column to remove hold up water in the chromatographic bed. Desorption was carried out using 350 liter of 55:45 composition of methanol:butanol mixture. During desorption 80 liter of water was collected separately followed by product fraction. Total 12.9 kg of TGS was recovered in 180 liter of elution phase as 7.2% w/v solution. The recovery was 99.2% and moisture content was 1.8% by Karl fisher method. This solution was then distilled under vacuum at 50 degree Celsius temperature to remove butanol and methanol where the moisture of 1.8% was removed as butanol-water azeotrope. Final mass was then crystallized from methylene dichloride to get 96% of TGS on the basis of feed. The purity of the crystallized product was 99.3% on HPLC. The remaining mother liquor was recycled in next cycle after solvent removal so as recover all TGS.

### Example 10

### Isolation and Crystallization of TGS-Acetate from Aqueous Solution of TGS-Acetate

The column was filled with 225 liter of SEPABEADS 207 (Mitsubishi Chemical Corporation, Japan) in stainless steel column of 310 mm diameter. The matrix was washed and equilibrated with water of pH 6.5. The matrix was loaded with 15 kg partially purified TGS-AC in aqueous-organic solution containing 95% water after extraction. Nitrogen was purged through the column to remove hold up water after draining the water and then followed by desorption using 350 liter of 55:45 composition of butanol:methanol mixture. During desorption 90 liter of water was collected separately followed by product fraction of 200 liter. Total 14.2 kg of TGS-AC was recovered in 200 liter of elution phase as 7.5% w/v solution at of 94.7% recovery and moisture content of 3.2% by Karl fisher method. This solution was further processed as described in example 2, to get 13.7 kg of TGS-AC.

### Example 11

### Capture, Tertiary Amide Removal, and Hydrolysis of 6-O-Protected TGS, and Recovery of TGS

2.0 liter of neutralized reaction mass containing 21 gm of 6-O-protected TGS and other chlorinated sucrose derivatives, with inorganic and organic salts and 0.5 Kg of tertiary amide passed through a 25 mm diameter borosilicate glass column equipped with stainless steel flow adapters at the two end and filled with pre-equilibrated 0.40 L of SEPABEADS SP700 (Mitsubishi Chemical Corporation, Japan) The feed was charged using a pump at rate of 1.2 liters/hour followed by washing with deionized water to remove unabsorbed mass. The adsorbed 6-O-protected TGS and other chlorinated sucrose derivatives was eluted from adsorbent matrix using 1.0 liter of hydrolyzing elution mobile phase containing 70% of methyl alcohol, 2.5% of ammonia and remaining portion as water. All unbound, wash and hydrolyzed elution fraction was analyzed for 6-O-protected TGS as TGS and tertiary amide by HPLC and GC respectively. The 6-O-protected chlorinated sucrose was also analyzed by TLC. The unbound and wash fraction does not show any TGS on HPLC and TLC indicating 100% adsorption efficiency of adsorbent for 6-O-protected TGS from reaction mass. The GC result shows total 0.496 Kg of tertiary amide in unabsorbed fractions. The elution fraction of 0.5 liter shows total 20.8 gm of TGS without any 6-O-protected TGS on TLC. The GC results show absence of tertiary amide in elution fraction. The yield related to the TGS and tertiary amide content of the starting reaction mass amounts to 99.04% in elution and 99.2% in unabsorbed fraction.

### Example 12

### Scale up of Capture, Tertiary Amide Removal, and Hydrolysis of 6-O-Protected TGS, and Recovery of TGS

1400 liter of neutralized reaction mass containing 12.0 Kg of 6-O-protected TGS and 350 kg of DMF was fed to the 230 liter SEPABEADS SP700 (Mitsubishi Chemical Corporation, Japan) packed in a 300 mm dia stainless steel column to get 3.0 meter bed height. The feed was charged using dosing pump at rate of 6.0 liter per minute followed by washing with deionized water. Unbound and wash fraction shows absence of 6-O-protected TGS on HPLC and TLC. Thus the adsorption efficiency was 100% for said TGS precursor. The adsorbed 6-O-protected TGS was then eluted with 850 liters of hydrolyzing elution mobile phase so as get 6-O-deprotected TGS. The composition of hydrolyzing elution mobile phase was 80:2.5:17.5 of methanol:ammonia:water. The hydrolyzed 6-O-protected TGS was recovered in 450 liter of elution fraction as concentrated mass. The concentration of TGS in elution was 2.64% showing 11.89 kg of TGS. GC analysis of elution fraction shows absence of DMF. The GC analysis unabsorbed and wash fraction shows 347.8 kg of DMF. Recovery of TGS and DMF was 99.1% and 99.37%.

### Example 13

### Use of Expanded Bed Chromatography for the Process of Present Invention for Capture, Tertiary Amide Removal, and Hydrolysis of 6-O-Protected TGS, and Recovery of TGS

The process was carried out in expanded bed 1.0 liter of SEPABEADS SP207 (Miitsubhishi chemical corporation, Japan), in 5.0 cm diameter glass column equipped with stainless steel adaptors at both ends. The adsorbent was loaded with 5.0 liter of neutralized reaction mass in upward flow direction. The loaded neutralized chlorinated mass reaction contains 60 gm of 6-o-protected TGS and other chlorinated sucrose derivatives, with inorganic and organic salts and 1.2 Kg of tertiary amide solvent. The degree of expansion was kept at 1.5 during loading. Further the adsorbed matrix was washed with 2 bed volume of 0.1M sodium hydroxide solution (1 bed volume in expanded bed and 1 bed volume in packed bed mode). This was then followed by elution with hydrolyzing elution mobile phase. 3.6 bed volume of hydrolyzing elution mobile phase containing 80% isopropyl alcohol and 3.75% of ammonia was passed through the column and 1.2 bed volume of enriched fraction containing 6-O-deprotected TGS was collected. Rest of the mobile phase acts as regenerating mobile phase and was collected separately. Elution was carried out in downward direction in packed bed mode. Total 58 gm of TGS was recovered in 1.2 bed volumes of elution as 4.83% solution, shows no residual 6-O-protected TGS and DMF. The unabsorbed and wash fraction collected from top of the column shows 1.18 kg of tertiary amide as DMF. Recovery of TGS was 96.67% whereas DMF recovery was 98.3%.

### Example 14

### Reusability and Performance of Matrix

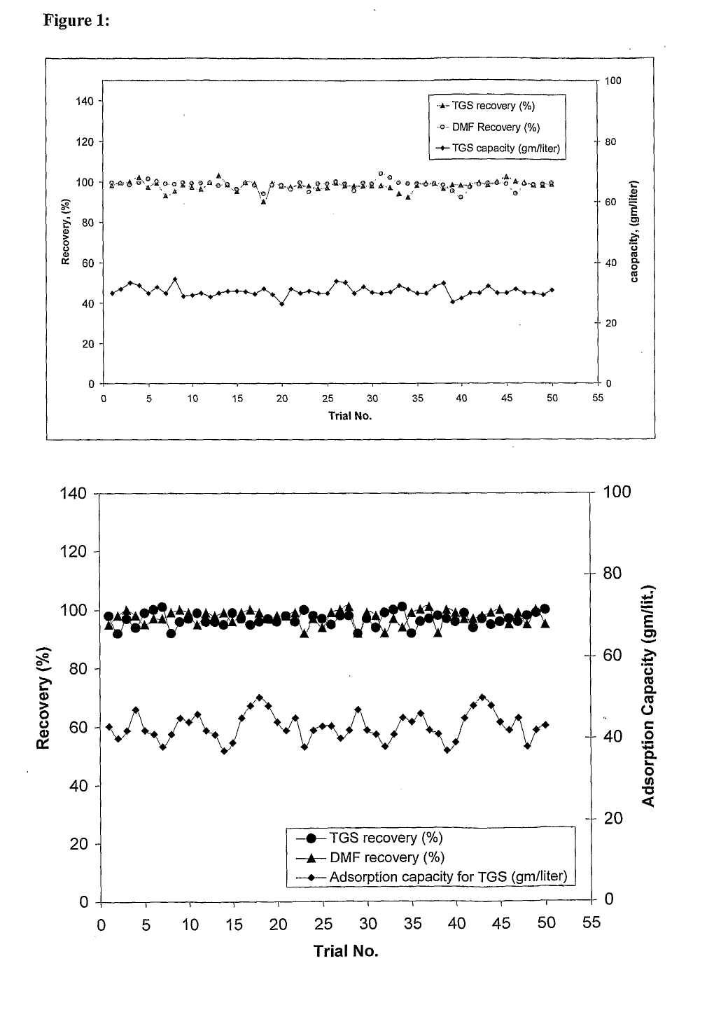

The reusability and performance in terms of purity and recovery of the product of the process of present invention is shown in FIG. 1 for 50 trials showing performance comparison of purification trials using SEPABEADS SP700 (Mitsubishi Chemical Corporation, Japan), for the process of the present invention in terms of matrix capacity, (gm/liter), TGS recovery, (%), and DMF recovery (%) as per example 6. The comparison shows that the Cleaning In place (CIP) of adsorbent matrix using regeneration mobile phase is efficient. Here the same matrix was used after regeneration using 95:1:5 methanol:ammonia:water composition as mobile phase. The matrix shows the consistent performance since after 50 trials and hence the reusability leading to improved economics of the process.

1. A process of separation and a further one or more of a process step comprising isolation, purification, concentration, de-watering, one or more of a chemical modification including de-esterification, and the like, of one or more of a chlorinated sucrose compound, the said chlorinated compound including a precursor as well as a derivative of a chlorinated sucrose compound, from a process stream, and comprising at least one or more of a following steps:
a. selective capture of the said one or more of a chlorinated sucrose compound on an adsorbent and the exclusion of other components of the said process stream by bringing the said process stream in contact with the said adsorbent, where the said adsorbent is not a silica gel or a porous or gel cation exchange resin,
b. selective elution of one or more of an adsorbed chlorinated sucrose compound from the said adsorbents, in an unchanged chemical form or a changed chemical form including a de-esterified form, individually or elution of a group of related chlorinated compounds, and where the said adsorbent is not a silica gel or a porous or gel cation exchange resin,
c. subjecting the eluant of step (b.) to one or more of a next process step for producing a product including a process of isolation and purification of a chlorinated sucrose compound.

2. A process of claim 1 wherein:
a. the said process stream or reaction mixture comprising a composition produced during the course of a process step of synthesis of or purification of a chlorinated sucrose or its precursor or its derivative, comprising a solution, with or without water, of reactants or products one of which at least comprises of one or more of (i) Glucose-6-ester including Glucose-6-acetate and Glucose-6-benzoate and the like (ii) Sucrose-6-ester including Sucrose-6-acetate and Sucrose-6-benzoate and the like (iii) 1-6-Dichloro-1-6-DIDEOXY-β-Fructofuranosyl-4-chloro-4-deoxy-galactopyranoside abbreviated as TGS, (iv) TGS-6-ester including TGS-6-acetate and TGS-6-benzoate and the like (v) Tetrachloro sucrose ester including Tetrachloro-6-acetate and Tetrachloro-6-benzoate and the like (vi) Tetrachloro sucrose, (vii) Dichloro sucrose ester including Dichloro-6-acetate and Dichloro-6-benzoate, (viii) Dichloro sucrose, (ix) inorganic salts, (x) organic salts, (xi) suspended solids, (xii) Tertiary amide, (xiii) soluble enzymes, (xiv) immobilized enzymes, (xv) penta acetyl sucrose, (xvi) sucrose alkyl 4,6-orthoacylate, (xvii) sucrose 2,3,6,3′,4′-penta ester, (xviii) Sucrose 6,4′-diesters, (xix) 4′,6-di-O-acetylsucrose, (xx) 6-O-acetylsucrose, (xxi) 2,3,6,3′-sucrose tetraacetate, (xxii) sucrose alkyl 4, (xxiii) 6-orthoester, (xxiv) sucrose octaacylate, (xxv) sucrose heptaacylate, and sucrose hexaacylate, (xxvi) sucrose alkyl 4,6-orthoester, (xxvii) sucrose 4-ester, (xviii) TGS penta acylates including TGS penta acetate, penta propionate, penta butyrate, penta glutarate, penta laureate; (xix) products of caramelization and the like,
b. the said precursor of a chlorinated sucrose includes one or more of (i) glucose, (ii) sucrose, (iii) sucrose-6-ester including sucrose-6-acetate and sucrose-6-benzoate, (iv) TGS-6-ester including TGS-6-acetate and TGS-6-benzoate, (v) tetrachlororaffinose, (vi) penta acetyl sucrose, (vii) sucrose alkyl 4,6-orthoacylate, (viii) sucrose 2,3,6,3′,4′-penta ester, (ix) Sucrose 6,4′-diesters, (x) 4′,6-di-β-acetylsucrose, (xi) 6-O-acetylsucrose, 2,3,6,3′-sucrose tetraacetate, (xii) sucrose alkyl 4,6-orthoester, (xiii) sucrose octaacylate, (xiv) sucrose heptaacylate, and sucrose hexaacylate, (xv) sucrose alkyl 4,6-orthoester, (xvi) sucrose 4-ester; and the like,
c. the said derivative of chlorinated sucrose includes their pentacylate further including a TGS penta acylate which further includes TGS penta acetate, TGS penta propionate, TGS penta butyrate, TGS penta glutarate, TGS penta laureate and the like,
d. the said adsorbent matrix is capable of interacting with a chlorinated sucrose compound, and comprising one or more of following: (i) a non sulfonic resin (ii) non ionic resin (iii) an anion exchange resin (iv) having a surface and/or surface group, which has interacting ability with chlorinated sucrose (v) which is rigid and porous, (vi) in the form of a membrane, (vii) has synthetic or natural polymeric base matrix, (viii) has a synthetic base matrix of polystyrene, divinylbenzene (PSDVB), polymethacrylates, polyacrylamide and the like, (ix) has natural polymeric base matrix of agarose, cellulose, chitosan, dextran and the like, (x) is crosslinked, (xi) a modified silica with aromatic and/or aliphatic moiety as substituted group having C2 to C18 carbon atoms, (xii) (xiii) has interacting group which is a part of base matrix or grafted on the base matrix by known activation chemistry, (xiv) the said interacting group is unsaturated or saturated aliphatic and/or an aromatic moiety of a C1-C18 carbon molecules, (xv) has the interacting group is halogen atom, (xvi) the interacting group is cyano, diol or amino, (xvii) has the interacting group which has different interacting ability or affinity or binding strength with different chlorinated sucrose, (xviii) microporous, macroporous, mesoporous, gigaporous, supermacroporous or throughporous, (xix) a mixed mode or anion exchange matrix based on one or more than one of a synthetic or natural polymeric matrix and having amino (primary, secondary or tertiary) or imino moiety, (xx) a matrix based on one or more of a polymer comprising PSDVB, polymethacrylates, polyacrylamide, a natural polymer and combinations thereof having hydroxyl or diol group, (xxi) a hydrophobic group,
e. the mobile phase used for equilibration, washing, elution and regeneration in both purification and polishing contains one or more of following: (i) water at neutral pH of 7, (ii) acidified water at pH below 7, (iii) alkaline water at pH above 7 and (iv) one or more of an alcohol including methanol, ethanol, isopropanol, butanol and the like, (v) acetonitrile, (vi) chlorinated organic solvents including chloroform, dichloromethane, dichloroethane and the like, (vii) toluene, (viii) one or more of an ester including butyl acetate, ethyl acetate and the like, (ix) one or more of a ketone including acetone, methyl isobutyl ketone and the like, (x) one or more of a ion-pairing agents or agents and one or more of an affinity and/or binding strength modifier including phosphoric acid, acetic acid, pentane sulphonic acid, trifluoro acetic acid, triphenylamine and a combination thereof, (xi) one or more of a buffer, including a citrate buffer, a phosphate buffer, an acetate buffer, a phosphate citrate buffer, a citrate-acetate buffer, borate buffer, carbonate buffer and the like, (xii) one or more of organic or inorganic salts such as but not limited to sodium chloride, sodium acetate, sodium carbonate, potassium phosphate, potassium citrate, potassium carbonate, potassium acetate, ammonium sulphate, ammonium chloride (xiii) one or more of organic or inorganic acid or base such as but not limited to acetic acid, citric acid, tartaric acid, hydrochloric acid, phosphoric acid, sulphuric acid, sodium hydroxide, potassium hydroxide, triethylamine, polyethylenimine, etc. (xiv) and any suitable combination of one or more of (i) to (xiii) mentioned above; chosen to achieve the desired affinity and/or interaction ability of the mono-chlorinated, di chlorinated, tri chlorinated and tetra chlorinated compounds with the adsorbent matrix as required,
f. further the mobile phase used for equilibration, washing, elution and regeneration comprises one or more of those mentioned in step (e) of this claim above and applied to the adsorbent in one or more of a method comprising continuing elution without any change in the eluant in an unchanged manner as in isocratic elution, or step wise manner or in a changing manner over a period of time and over any volume of liquid, as in ‘step gradient’ or any suitable ‘continuous gradient’ elution, or any combinations thereof.

3. A process of claim 1 comprising one or more of a step of:
a. using an adsorbent having on their matrix one or more of a interacting chemical group or a ligand selective for trichloro and tetrachloro derivatives of sucrose, further comprising a benzyl or a phenyl group and the like, to adsorb trichloro and tetrachloro derivatives of sucrose on the adsorbent,
b. washing away unadsorbed components of the feed, if any, comprising one or more of DMF, inorganic salts, organic salts, organic solvents, caramelization products and the like by a mobile phase comprising one or more of following: (i) water at neutral pH of 7, (ii) acidified water at pH below 7, (iii) alkaline water at pH above 7 and (iv) one or more of an alcohol including methanol, ethanol, isopropanol, butanol and the like, (v) acetonitrile, (vi) chlorinated organic solvents including chloroform, dichloromethane, dichloroethane and the like, (vii) toluene, (viii) one or more of an ester including butyl acetate, ethyl acetate and the like, (ix) one or more of a ketone including acetone, methyl isobutyl ketone and the like, (x) one or more of a ion-pairing agents or agents and one or more of an affinity and/or binding strength modifier including phosphoric acid, acetic acid, pentane sulphonic acid, trifluoro acetic acid, triphenylamine and a combination thereof, (xi) one or more of a buffer, including a citrate buffer, a phosphate buffer, an acetate buffer, a phosphate citrate buffer, a citrate-acetate buffer, borate buffer, carbonate buffer and the like, (xii) one or more of organic or inorganic salts such as but not limited to sodium chloride, sodium acetate, sodium carbonate, potassium phosphate, potassium citrate, potassium carbonate, potassium acetate, ammonium sulphate, ammonium chloride (xiii) one or more of organic or inorganic acid or base such as but not limited to acetic acid, citric acid, tartaric acid, hydrochloric acid, phosphoric acid, sulphuric acid, sodium hydroxide, potassium hydroxide, triethylamine, polyethylenimine, etc. (xiv) and any suitable combination of one or more of (i) to (xii) mentioned above; chosen to achieve the desired affinity and/or interaction ability of the mono-chlorinated, di chlorinated, tri chlorinated and tetra chlorinated compounds with the adsorbent matrix as required, the mobile phase constituted in a manner suitable for washing of unadsorbed components of the feed, if any, comprising one or more of DMF, inorganic salts, organic salts, organic solvents, caramelization and dictated by the adsorbent matrix used for purification,
c. washing or eluting with another suitably constituted mobile phase, from the group mentioned in claim 2(e), and optionally isolating dichloro and monochloro derivatives in the mobile phase applied in a manner described in claim 2(f),
d. isolating the trichloro and tetrachloro derivatives in pure fractions by selective elution using one or more of a suitably constituted mobile phase from the group mentioned in claim 2(e) and applied in a manner of claim 2(f).

4. A process of claim 1 comprising one or more of a step of:
a. using an adsorbent having on its matrix one or more of an interacting chemical group or a ligand selective for all chlorinated sucrose derivatives, the said sucrose derivatives comprising one or more of tetrachoro, trichloro, dichloro and monochloro derivatives of sucrose, the said ligands comprising non-ionic or cationic aliphatic and/or cationic aromatic compounds further comprising a halogen and the like, further comprising a bromine and the like, to adsorb one or more of a tetrachloro, trichloro dichloro or a monochloro derivative of sucrose, followed by,
b. washing away unadsorbed components of the feed, if any, comprising one or more of DMF, inorganic salts, organic salts, organic solvents, caramelization products and the like by continuing elution, the said eluant being suitably constituted mobile phase, from the group mentioned in claim 2(e), and applied in a manner described in claim 2(f),
c. selective elution of one or more of a chlorinated sucrose in a pure fraction separate from each other, the said eluant being suitably constituted mobile phase, from the group mentioned in claim 2(e), and applied in a manner described in claim 2(f).

5. A process of claim 1 comprising one or more of a step of:
a. using an adsorbent having on their matrix one or more of an interacting chemical group or a ligand selective for a trichloro derivative of sucrose comprising one or more of an amino group, or imino group and the like to adsorb a trichloro derivative of sucrose,
b. washing away unadsorbed components of the feed, if any, comprising one or more of DMF, inorganic salts, organic salts, organic solvents, caramelization products and the like by continuing elution, the said eluant being suitably constituted mobile phase, from the group mentioned in claim 2(e), and applied in a manner described in claim 2(f),
c. washing out dichloro and monochloro derivatives of sucrose out of the column and optionally collecting them separately, the said washing solution being suitably constituted mobile phase, from the group mentioned in claim 2(e), and optionally isolating dichloro and monochloro derivatives in the mobile phase applied in a manner described in claim 2(f),
d. eluting out the trichloro derivative of sucrose as a pure fraction, the said eluant being suitably constituted mobile phase, from the group mentioned in claim 2(e), and applied in a manner described in claim 2(f).

6. A process of claim 1 wherein a process stream containing one or more of a chlorinated sucrose compound, preferably with 5% or more in concentration in the said process flow, is concentrated as well as dewatered comprising one or more of a step of:
a. adsorbing a chlorinated sucrose compound onto an adsorbent by loading the column by draining the column under gravity, or using a suitable drive such as pump or pressurized gas, allowing excess liquid phase to drain away,
b. followed by purging with gas to remove almost all free water, or water-solvent mixture, present in the matrix bed, and
c. eluting the adsorbed molecule with a solvent comprising water free organic solvent or mixture of solvents selected from the group but not limited to (i) one or more of an alcohol including methanol, ethanol, isopropanol, butanol and the like, (ii) acetonitrile, (iii) chlorinated organic solvents including chloroform, dichloromethane, dichloroethane and the like, (iv) toluene, (v) one or more of an ester including butyl acetate, ethyl acetate and the like, (vi) one or more of a ketone including acetone, methyl isobutyl ketone and the like.

7. A process of claim 1 wherein de-esterification is integrated in a process of chromatography of a solution or a process stream containing a chlorinated sucrose ester comprising steps of:
a. eluting a column having adsorbent on which a chlorinated sucrose ester is adsorbed with an eluant capable of eluting as well as de-esterifying the chlorinated sucrose ester to respective chlorinated sucrose in the column itself, the said eluant being suitably constituted mobile phase, from the group mentioned in claim 2(e), and applied in a manner described in claim 2(f),
b. isolating and purifying the chlorinated sucrose from the eluted out solution.

8. A process of claim 1 wherein the process stream comprises one or more of:
a. a chlorination reaction mixture applied before or after de-protection of the neutralized chlorinated sucrose derivatives,
b. a chlorination reaction mixture applied before or after the removal of the tertiary amide,
c. removal of tertiary amide and salts from said chlorinated sucrose derivatives,
d. removal of tertiary amide and salts from said chlorinated sucrose derivatives before or after the removal of salts (organics an/or inorganics) partially or completely,
e. a reaction mixture at any stage after partial purification of chlorinated sucrose derivatives through any of the other operations such as extraction, chromatography, crystallization, Distillation, and the like,
f. as a substitute to traditional column chromatography,
g. to purify or isolation of any sucrose intermediate compound used for the preparation of the said chlorinated sucrose derivatives,
h. for further purification of isolated TGS or its precursors or derivatives by subjecting the solution for column chromatography of this invention, and the like.

9. A process of claim 1 wherein the process is carried out in one or more of a mode comprising in single or in multiples or a combination of a batch mode, a continuous mode, an expanded bed, a fluidized bed, a liquid solid circulating fluidized bed (LSCFB), a simulated moving bed (SMB), a moving bed, an improved simulated moving bed (ISMB), a centrifugal chromatography, an annular chromatography; adsorption being preferably performed with a packed bed chromatographic column or expanded bed chromatographic column, which comprises packing the column with a suitable adsorbent and passing the reaction mass and mobile phase/s through the column.

10. A process of claim 7, as, applied to one or more of a process stream:
a. applied before or after de-protection of the chlorinated sucrose derivative/s,
b. applied after enzymatic or chemical de-protection of the purified or partially purified chlorinated sucrose derivative/s,
c. applied before or after the removal of the tertiary amide solvent,
d. applied at any stage after partial purification of chlorinated sucrose derivative/s through any of the other operations such as extraction, chromatography and the like,
e. as a substitute to traditional extraction and distillation to remove water,
f. applied after purification or isolation of any chlorinated sucrose intermediate compound used for the preparation of the desired chlorinated sucrose derivative/s.

11. A process of claim 1 wherein the process is carried out in the range of 0 to 80 degree Celsius, preferably at prevalent ambient temperature.
